(19) United States
(12) Patent Application Publication
(10) Pub. No.: US 2025/0186337 A1
(43) Pub. Date: Jun. 12, 2025

(54) CHEWABLE ORAL CONTRACEPTIVE

(71) Applicant: CHEMO RESEARCH, S.L., Madrid (ES)

(72) Inventors: Enrico COLLI, Madrid (ES); Jean-Daniel BONNY, Lugano (CH); Sandra UREK, León (ES); Maria Ángeles PÉREZ DE LA CRUZ, Azuqueca de Henares (Guadalajara) (ES)

(21) Appl. No.: 18/842,742

(22) PCT Filed: Feb. 28, 2023
(86) PCT No.: PCT/EP2023/055017

§ 371 Date: Aug. 29, 2024

(30) Foreign Application Priority Data
Mar. 1, 2022 (EP) 22382182.8

(51) Int. Cl. A61K 9/00; A61K 9/20; A61K 31/585

(52) U.S. Cl. CPC A61K 9/0056; A61K 9/2009; A61K 9/2018; A61K 9/2054; A61K 31/585

(57) ABSTRACT

The present invention pertains to the field of oral pharmaceutical compositions and their use for providing contraception. More specifically, the present invention relates to oral chewable tablets of drospirenone, their use for providing contraception to an individual and a kit comprising daily dosage units of such oral chewable tablets.

### FIELD OF THE INVENTION

The present invention pertains to the field of oral pharmaceutical compositions and their use for providing contraception. More specifically, the present invention relates to oral chewable tablets of drospirenone, their use for providing contraception to an individual and a kit comprising daily dosage units of such oral chewable tablets.

### BACKGROUND OF THE INVENTION

Drospirenone (DRSP) is a derivative of spironolactone which has progestomimetic, antimineralocorticoid and antiandrogenic activity. Drospirenone as a contraceptive ingredient is available in oral combined pills such as those marketed under the name of Yasmin® (3 mg DRSP/30 μg ethinyl estradiol), Yaz® (3 mg DRSP/20 μg ethinyl estradiol) and Yasminelle® (3 mg DRSP/20 μg ethinyl estradiol). These pills comprise ethinyl estradiol which acts to increase the ovulation inhibitory effect of drospirenone and to ensure contraception and cycle control. In the EU and USA, DRSP has been approved as a POP (progestin-only-pill) for contraception in women of reproductive age (Slinda™/Slynd™). The approved posology is one active tablet (DRSP 4 mg) to be swallowed daily during the first 24 days and one inactive tablet to be swallowed daily during the 4 following days.

Although Slynd™ contains 33% more drospirenone than Yaz®/Yasminelle® combined oral contraceptive (3 mg DRSP+0.02 mg ethinylestradiol) products, the extent of systemic drospirenone exposure at steady state is about 32% less with the 4 mg formulation. Combined with a reduced Cₘₐₓ, the pharmacokinetic profile of Slynd™ provides similar efficacy as the combined products and enhanced safety, widening the group of women able to use this contraceptive method. It has been suggested (Richter, 2020) that metabolic pathways of drospirenone can be inhibited by ethinyl estradiol, resulting in higher DRSP plasma concentrations in DRSP/EE formulations than in a drospirenone alone formulation.

Additionally, whilst POPs are a reliable and beneficial mode of providing contraception, the actual contraceptive active ingredient itself is not the only decisive factor when it comes to providing efficient and reliable contraception to a large number of females. Patient compliance is also one of the main factors for efficacy of oral contraceptives, the most common reason for failure of the oral contraceptive being that it is not being taken regularly. Different solutions have been offered to improve patient compliance with contraceptive regimens, such as for example rods that can be inserted under the skin, intramuscular and intrauterine devices, vaginal rings, or subdermal injections of a depot every three months. However, some of these techniques require either surgical intervention, or an injection via hypodermic needle, which are both disfavoured by many patients. The by far most accepted mode of administration of a contraceptive is still the oral administration as it does not require any medical intervention. Further benefits of the oral contraceptives are that they can be carried on the person, allowing flexibility, and that they can be purchased also as single monthly strips thus not requiring a higher upfront payment, like the longer lasting solutions. Prior proposed solutions to the compliance problem for oral contraceptives focus primarily on optimizing the packaging, rather than on changes to the dosage form. However, a substantial amount of people does not feel comfortable swallowing tablets or need liquid to do so, these being some of reasons for a delay in taking the contraceptive on time. Different forms of packaging of the oral contraceptive cannot remedy this.

There is therefore still a need to provide for an improved mode of administration of oral contraceptive POPs that combine the above-mentioned benefits with ease of administration leading to increased patient compliance and thus to more reliable contraception.

One way of overcoming the above problems are oral chewable contraceptives, such as Ovcon 35, the first FDA approved contraceptive tablet that can be chewed and swallowed, containing a progestin (norethindrone) and an estrogen (ethinyl estradiol). Another oral chewable contraceptive is disclosed in U.S. Pat. No. 6,667,050, which comprises a progestin alone or in combination with an estrogen, the preferred progestin being norethindrone.

However, to date no POP has been approved as an oral chewable contraceptive, one main hurdle being the development of a contraceptive tablet that upon chewing provides the same reliable contraceptive effect as an oral tablet that is being swallowed. The chewing process leads to partial disintegration of the tablet already in the mouth, whereas the swallowed tablet stays intact until it reaches the gastrointestinal tract. The release of the active ingredient thus needs to be controlled to ensure that no undesirable high plasma concentration peak for the active ingredient is reached. High plasma concentrations are not desired in patients using drospirenone because of a correlation between high Cₘₐₓ and certain undesirable side effects as well as poor general tolerance when hormonal levels fluctuate too much every day.

In view of the known benefits of POPs, specifically POPs comprising drospirenone as the active ingredient, over combined oral contraceptives, as well as the still unresolved patient compliance problems leading to less efficacious contraception, there is a need for a novel POP comprising drospirenone as the active ingredient that can overcome these disadvantages.

### SUMMARY OF THE INVENTION

The present invention therefore relates in one aspect to an oral chewable tablet comprising a composition comprising 3.4 mg-3.7 mg, preferably 3.5 mg of drospirenone, wherein the tablet has a dissolution profile characterized in that 20-40%, of the drospirenone initially present in said composition is dissolved within 30 minutes when the composition is subjected to an in vitro dissolution test in 900 ml of water with 0.6% polysorbate using USP apparatus 2 (paddles) at 100 rpm, preferably at a temperature of 37° C.±0.5° C., the percentages of drospirenone being related to the amount of drospirenone initially present in said tablet.

In a preferred embodiment at least 75% of said drospirenone is dissolved after 4 hours. In another preferred embodiment, optionally in combination with the above, at least 50%, preferably at least 60%, 65% or 70% of said drospirenone is dissolved in a time range from about 3 hours to about 4 hours.

In one embodiment the tablet does not comprise any estrogen, preferably said tablet comprises drospirenone as the only contraceptive ingredient.

In a further embodiment the oral chewable tablet comprises drospirenone as the only contraceptive ingredient, preferably 3.4 mg-3.7 mg, preferably 3.5 mg of drospirenone, wherein said tablet further has a hardness of 15 N to 100 N, preferably of 30 N-75 N, more preferably of 40 N-65 N.

The present invention also relates in a second aspect to an oral chewable tablet comprising a composition comprising 3.4 mg-3.7 mg, preferably 3.5 mg of drospirenone, wherein said tablet further has a hardness of 15 N to 100 N, preferably of 30 N-75 N, more preferably of 40 N- 65 N and wherein the tablet does not comprise any estrogen. In a preferred embodiment, this oral chewable tablet is characterized by the dissolution profile and/or pharmacokinetic features described for the other aspects of the invention.

In one embodiment the oral chewable tablet comprises drospirenone as the only contraceptive ingredient.

A third aspect of the invention relates to an oral chewable tablet comprising from 3.4 mg-3.7 mg, preferably 3.5 mg drospirenone, wherein the tablet is formulated such that the tablet provides a mean Cₘₐₓ of less than 30 ng/ml drospirenone, preferably of less than 25 ng/ml, in a subject after a single administration of the oral dosage form when the tablet is chewed or disintegrated in the oral cavity of the subject prior to swallowing.

In a further embodiment the invention relates to an oral chewable tablet comprising from 3.4 mg-3.7 mg, preferably 3.5 mg drospirenone, wherein the tablet is formulated such that the tablet provides a mean Cₘₐₓ between 14 ng/ml to 22.5 ng/ml in a subject after a single administration of the oral dosage form when the tablet is chewed or disintegrated in the oral cavity of the subject prior to swallowing.

In one embodiment of present invention said tablet has a dissolution profile characterized in that no more than 40-60%, preferably no more 40-50% of the drospirenone initially present in said composition is dissolved within 30 minutes when the tablet is crushed, and the crushed tablet is subjected to an in vitro dissolution test in 900 ml of water with 0.6% polysorbate, using USP apparatus 2 (paddles) at 75 rpm, preferably at a temperature of 37° C.±0.5° C., the percentages of drospirenone being related to the amount of drospirenone initially present in said tablet.

In a preferred embodiment at least 75% of said drospirenone is dissolved after 4 hours. In a preferred embodiment, optionally in combination with the above, at least 50%, preferably at least 60%, 65% or 70% of said drospirenone is dissolved in a time range from about 3 hours to about 4 hours.

In one embodiment the tablet does not comprise any estrogen, preferably said tablet comprises drospirenone as the only contraceptive ingredient.

The following embodiments relate to an oral chewable tablet of any of the first to third aspects of the invention.

In a further embodiment of the invention the drospirenone has a d50 particle size ranging from 10 μm to 60 μm, preferably a d50 particle size ranging from 20 μm to 50 μm.

In a further embodiment of the invention the oral chewable tablet further comprises one or more pharmaceutically acceptable excipients.

In one embodiment said pharmaceutically acceptable excipients comprise at least one binder and at least one filler, wherein:
(i) the amount of drospirenone accounts for 1% to 10% by weight,
(ii) the amount of the at least one binder accounts for 50% to 65% by weight and
(iii) the amount of the at least one filler accounts for 25% to 35% by weight, the percentages by weight being related to the total weight of the said tablet.

In a preferred aspect of this embodiment the oral chewable tablet further comprises at least one glidant and at least one lubricant wherein:
(i) the amount of the at least one glidant accounts for 0.2% to 6% by weight and
(ii) the amount of the at least one lubricant accounts for 0.2% to 2.5% by weight, the percentages by weight being related to the total weight of the said tablet.

In a preferred aspect of this embodiment the oral chewable comprises or consists of:
a) 3.4 mg-3.7 mg of drospirenone, preferably 3.5 mg of drospirenone;
b) at least one binder at 55% to 65% w/w, preferably about 60% w/w;
c) at least one filler at 25% to 35% w/w, preferably about 30% w/w;
d) at least one glidant 0.1% to 1% w/w; preferably about 0.5% w/w;
e) at least one lubricant 0.1% to 2.5% w/w; preferably about 0.5% w/w; and
f) optionally a flavouring agent 0.5% to 2% w/w; preferably about 1% w/w, the percentages by weight being related to the total weight of the said tablet.

In a further preferred embodiment, the at least one binder is microcrystalline cellulose, the at least one filler is anhydrous lactose, the at least one glidant is silicon dioxide and the at least one lubricant is magnesium stearate.

In another embodiment the oral chewable tablet comprises:
a) 3.4 mg-3.7 mg of drospirenone, preferably 3.5 mg;
b) microcrystalline cellulose 55% to 65% w/w; preferably about 60% w/w;
c) anhydrous lactose 25% to 35% w/w; preferably about 30% w/w;
d) silicon dioxide 0.1% to 1% w/w; preferably about 0.5% w/w;
e) magnesium stearate 0.1% to 2.5% w/w; preferably about 0.5% w/w; and
f) optionally, a flavouring agent 0.5% to 2% w/w; preferably about 1% w/w, the percentages by weight being related to the total weight of the said tablet.

In one embodiment of present invention the oral chewable tablet has a maximum diameter of between about 4.0 to 6.0 mm, more preferred of between about 4.5 to 5.5 mm, most preferred of about 5.0 mm. In a further embodiment of the invention the oral chewable tablet has a thickness of between about 1.9 mm to 3.6 mm, more preferred of between about 2.1 mm to 3.6 mm, most preferred of about 2.4 mm.

The present invention further relates to the use of the oral chewable tablet as described herein for providing contraception to an individual.

In one embodiment the present invention relates to the use of the oral chewable tablet as described herein for providing contraception to an individual, comprising administering one tablet once daily to said individual.

The present invention further relates to the use of the oral chewable tablet as described herein for providing contraception to an individual, comprising administering one tablet once daily to said individual for 24 to 28 consecutive days.

In one embodiment of the present invention the oral chewable tablet as described herein is administered during 24 consecutive days, optionally followed by the administration of 4 daily dosage units of a pharmaceutically acceptable placebo.

The present invention furthermore relates to a kit, preferably a contraceptive kit, comprising one or more packaging units, wherein each packaging unit comprises 24 to 28 active daily dosage units, each daily dosage unit comprising an oral chewable tablet according to any one of the preceding embodiments of the invention.

In a preferred embodiment of the method the oral dosage form does not comprise any estrogen. In an even more preferred embodiment drospirenone is the only active ingredient of the oral dosage form.

In an even more preferred embodiment of the method the oral dosage form is a chewable oral tablet as further described herein.

The present invention also relates to a method for providing contraception in a subject, comprising administering to the subject an oral dosage form comprising from 3.4 mg-3.7 mg, preferably 3.5 mg drospirenone, wherein the oral dosage form is chewed or disintegrated in the oral cavity of the subject prior to swallowing, and wherein the administering results in a mean Cₘₐₓ of less than 30 ng/ml drospirenone, preferably of less than 25 ng/ml drospirenone in the subject after a single administration of the oral dosage form. In a further embodiment the invention relates to a method for providing contraception in a subject, comprising administering to the subject an oral dosage form comprising from 3.4 mg-3.7 mg, preferably 3.5 mg drospirenone, wherein the oral dosage form is chewed or disintegrated in the oral cavity of the subject prior to swallowing, and wherein the administering results in a mean Cₘₐₓ between 14 ng/ml to 22.5 ng/ml in a subject after a single administration of the oral dosage form when the tablet is chewed or disintegrated in the oral cavity of the subject prior to swallowing.

The present invention furthermore relates to a method for providing contraception in a subject, comprising administering to the subject oral chewable tablet comprising 3.4 mg-3.7 mg, preferably 3.5 mg of drospirenone, wherein the tablet has a dissolution profile characterized in that 20-40%, of the drospirenone initially present in said composition is dissolved within 30 minutes when the composition is subjected to an in vitro dissolution test in 900 ml of water with 0.6% polysorbate using USP apparatus 2 (paddles) at 100 rpm, preferably at a temperature of 37° C.±0.5° C., the percentages of drospirenone being related to the amount of drospirenone initially present in said tablet.

In a preferred embodiment at least 75% of said drospirenone is dissolved after 4 hours.

In a preferred embodiment at least 50%, preferably at least 60%, 65% or 70% of said drospirenone is dissolved in a time range from about 3 hours to about 4 hours.

In another preferred embodiment of said method the tablet does not comprise any estrogen, preferably said tablet comprises drospirenone as the only contraceptive ingredient.

### BRIEF DESCRIPTION OF THE DRAWINGS

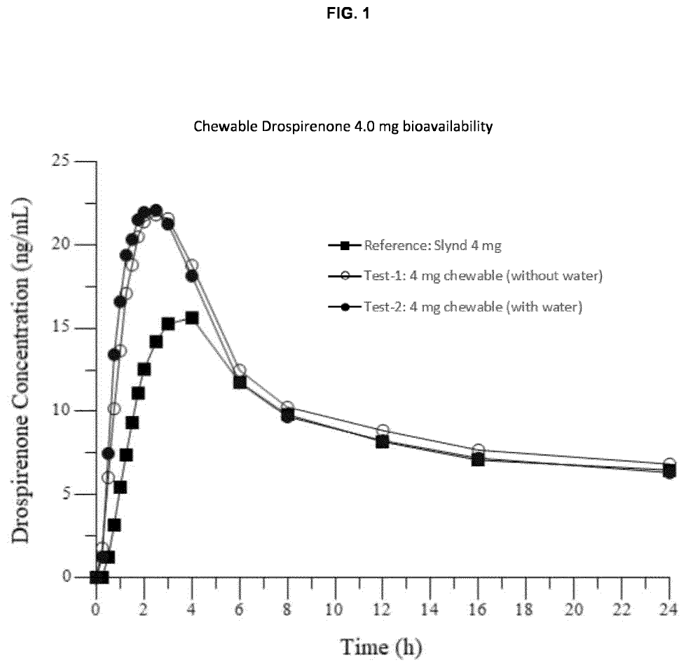

### FIG. 1: Bioavailability of Chewable Drospirenone 4.0 mg

FIG. 1 shows the comparison between the bioavailability of Drospirenone 4.0 mg chewable tablet using different modes of administration versus the reference product Slynd™ as further described in Example 1: chewed and swallowed without water (Test1) and chewed and swallowed with water (240 mL) (Test 2).

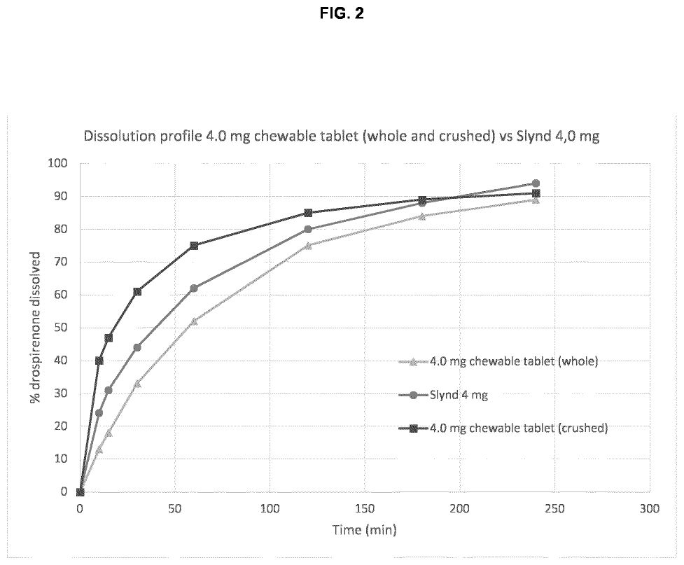

### FIG. 2: Comparison In Vitro Dissolution Profiles of Chewable Drospirenone 4.0 mg (Whole and Crushed) vs Slynd™

FIG. 2 shows a comparison of the dissolution profile of the 4 mg chewable tablet (whole and crushed) versus Slynd™. Testing conditions: 900 ml of water and 0.6% polysorbate, 75 rpm, apparatus II (paddles) as described further in Example 3.

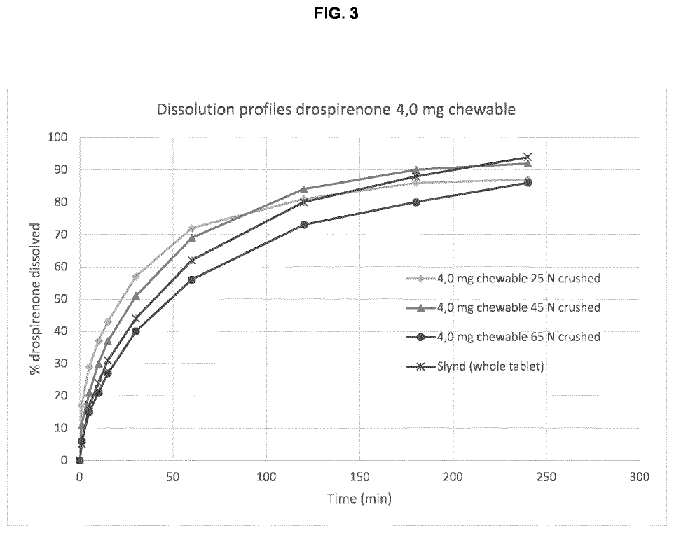

### FIG. 3: In Vitro Dissolution Profiles of Chewable Drospirenone 4.0 mg

FIG. 3 shows in vitro dissolution profiles in 900 mL water with 0.6% polysorbate, apparatus 2 (paddles), 75 rpm for chewable tablets comprising 4 mg of drospirenone with different hardness of 25 N, 45 N and 65 N tested as crushed tablets in comparison to Slynd™ (tested as whole tablet) and drospirenone 4 mg chewable tablets used in the bioequivalence study (tested as crushed tablet) as described further in Example 3.

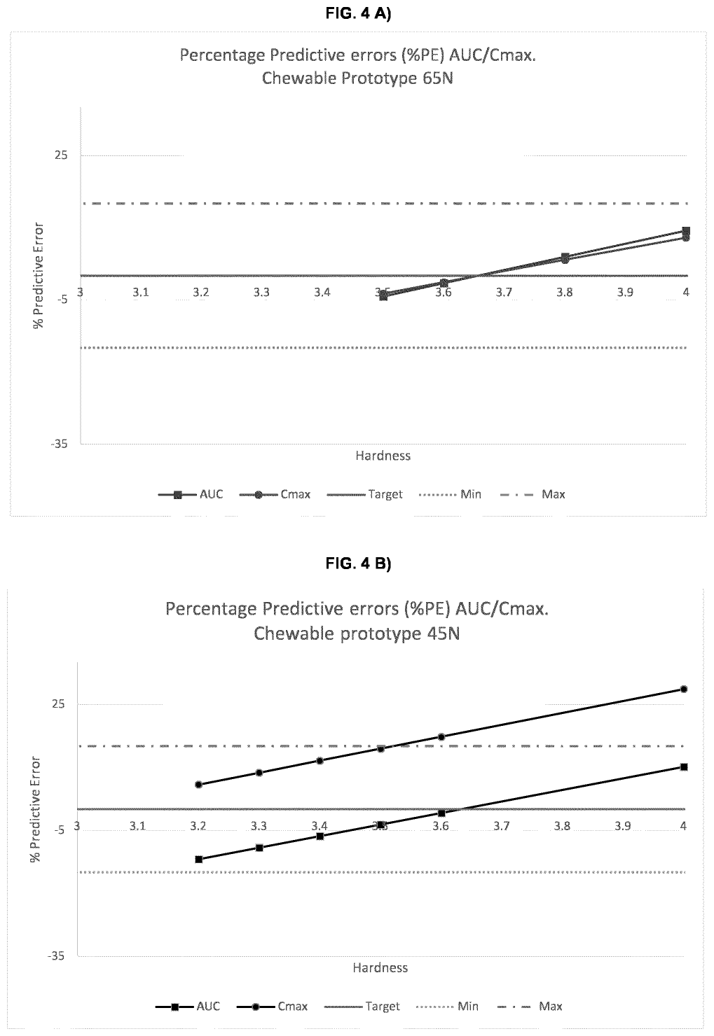

### FIG. 4: IVIVC Simulation for Prototype With 65 N Hardness and With 45 N Hardness

Percentage of predictive error (% PE) obtained for AUC₀₋₇₂ₕ and Cₘₐₓ values for 4.0 mg drospirenone chewable tablets with 65 N hardness (FIG. 4A) and with 45 N hardness (FIG. 4B) after simulation of the PK profile at different doses using the dissolution profiles, IVIVC developed and comparing to batch.

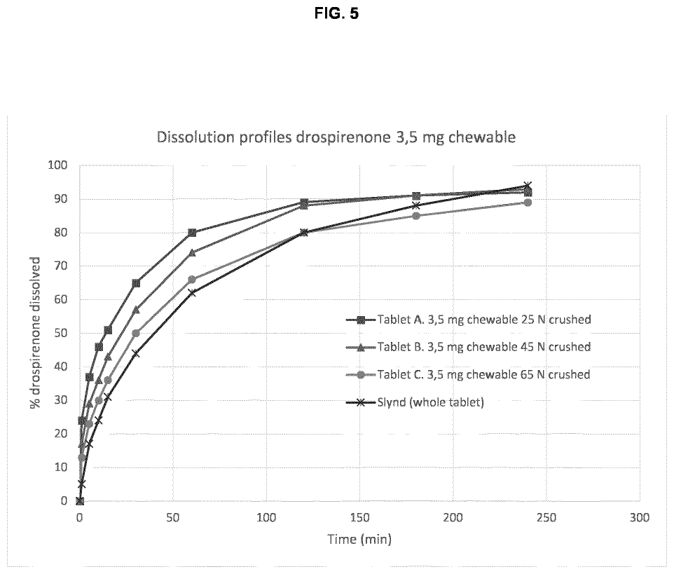

### FIG. 5: In Vitro Dissolution Profiles of Chewable Drospirenone 3.5 mg

FIG. 5 shows the first in vitro dissolution profiles for chewable tablets comprising 3.5 mg of drospirenone with different hardness of 25 N, 45 N and 65 N in comparison to Slynd™ as described further in Example 5.

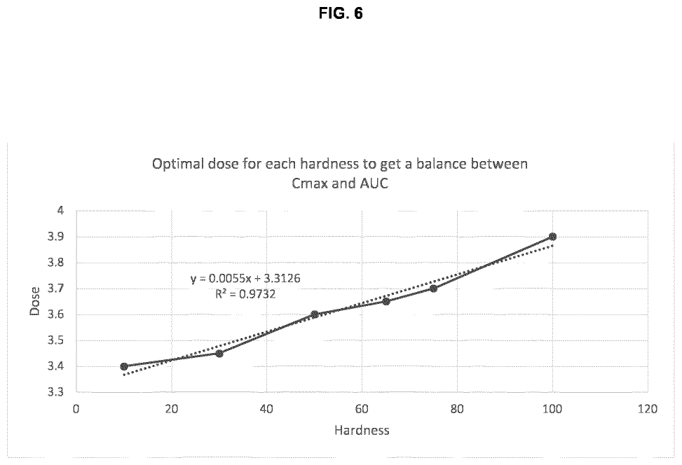

### FIG. 6: Relationship Between Dosage and Hardness on Bioequivalence

FIG. 6 shows the approximate relationship between the dose and the hardness to have an expected right balance for Bioequivalence of AUC₀₋₇₂ₕ and Cₘₐₓ between the chewable formulation and Slynd™.

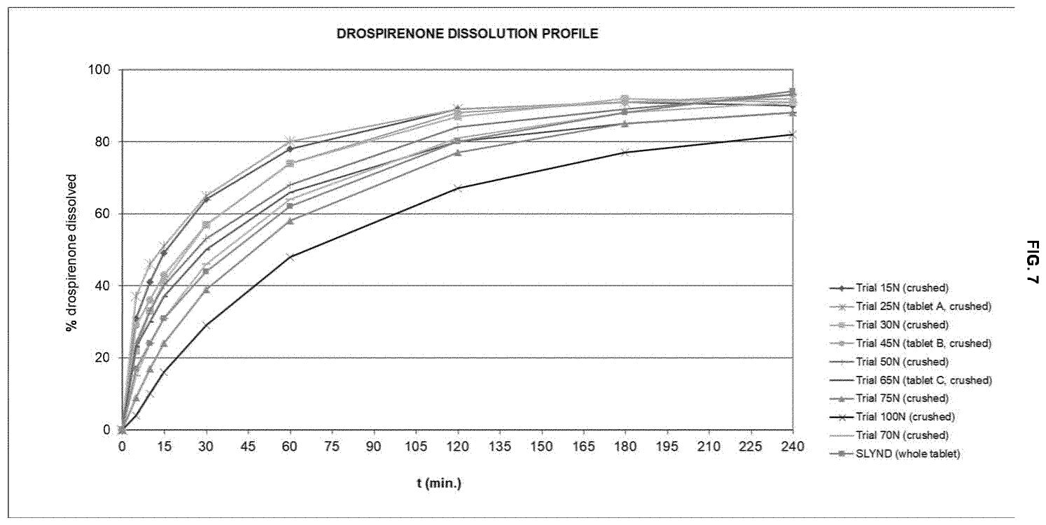

### FIG. 7: In Vitro Dissolution Profiles

FIG. 7 shows the in vitro dissolution profiles for chewable tablets comprising 3.5 mg of drospirenone with different hardness from 15 N to 100 N in comparison to Slynd™ as described further in Example 8.

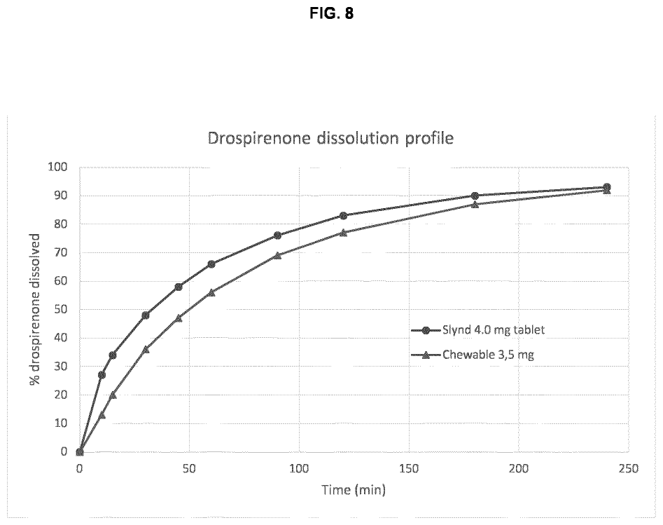

### FIG. 8: Comparative Dissolution Profiles Between Slynd™ 4 mg Tablets and 3.5 mg Chewable Tablets

FIG. 8 shows the comparative dissolution profiles in 900 mL water with polysorbate, apparatus 2 (paddles), 100 rpm, for Slynd™ 4 mg tablets and drospirenone 3.5 mg chewable tablets as described further in Example 9.

### DETAILED DESCRIPTION OF THE INVENTION

### Definitions

As used herein, the term “active daily dosage unit” or “daily dosage unit” refers to physically discrete units suitable as unitary dosage which can be administered to a subject to provide the required amount of active ingredient, such as drospirenone.

As used herein, the term “chewable” refers to a tablet that upon chewing in the mouth can be crushed or ground down into smaller pieces that allow easier swallowing of the tablet.

As used herein “crushed tablet” refer to tablets broken into small pieces by using a device, such as for example an artificial jaw, mimicking the chewing function and thus leaving the tablets in a broken state as they would be after having been chewed and cut in the mouth of a subject.

As used herein, the term “contraceptive kit” refers to a kit that is serving to prevent pregnancy when administered according to the instructions and in a daily effective amount to a female patient.

As used herein a “contraceptive method” relates to a method for preventing pregnancy.

As used herein, the term “drospirenone” refers to drospirenone itself, i.e. the chemical entity identified by the CAS registry Number 67392-87-4, solvates of drospirenone, and derivates or prodrugs of drospirenone. Drospirenone may be prepared by well-known methods described in the prior art, for example, described in U.S. Pat. No. 4,129,564, WO9806738, EP11746101 or W02006061309. The method described in W02006061309 may be particularly suitable for preparing drospirenone.

As used herein the term “hardness” relates to the force required to break or induce fractures in a tablet in diametrical compression test. Normally, for a given batch 10 samples are measured. However, depending on the batch size manufactured, and mostly at laboratory scale, the number of samples may also be 5 or even 3.

As used herein with respect to the dosage form of the invention, the term “oral”, “oral dosage form”, “oral pharmaceutical dosage form”, “oral administration”, “oral compositions” “oral pharmaceutical compositions”, “oral contraceptive compositions”, “oral tablets”, “oral capsules”, “orally ingested”, “orally”, “oral route” and the like all refer to any method of administration through the mouth.

As used herein “progestogen-only contraceptive”, or “progestogen-only pill” (also known as “POP”) means a pill or a contraceptive which comprises progestogens as sole contraceptive active ingredients and does not comprise any estrogen.

As used herein, the term “subject” refers to a nonpregnant female of child-bearing age i.e. from the puberty to the menopause seeking contraception. Women of child-bearing age also include women in perimenopause.

As used herein a “therapeutically effective amount” refers to an amount effective, at dosages and for periods of time necessary to achieve the desired therapeutic result, such as one or more of the following therapeutic results, such as a significant delay of the onset or progression of the disease; or a significant reduction of the severity of one or more symptoms. A therapeutically effective amount is also typically one in which any toxic or detrimental effect of the active ingredient or pharmaceutical composition is outweighed by the therapeutically beneficial effects.

To date no POP has been approved as a chewable contraceptive, one main hurdle being the development of a contraceptive tablet that upon chewing provides the same reliable contraceptive effect as an oral tablet that is being swallowed. The chewing process leads to partial disintegration of the tablet already in the mouth, whereas the swallowed tablet stays intact until it reaches the gastrointestinal tract. The release of the active ingredient thus needs to be controlled to ensure that no undesirable high plasma concentration peak for the active ingredient is reached. High plasma concentrations are not desired in patients using drospirenone because of a correlation between high Cₘₐₓ and certain undesirable side effects as well as poor general tolerance when hormonal levels fluctuate too much every day. The decrease of drospirenone Cₘₐₓ is desirable to reduce or avoid undesirable side-effects, in particular those related to plasma level of potassium.

The aim of the present invention was therefore to develop a chewable oral contraceptive comprising drospirenone, wherein the oral contraceptive does not contain any estrogen, preferably wherein drospirenone is the only active ingredient, and showing bioequivalence to drospirenone 4.0 mg tablets which corresponds to commercially available oral contraceptive Slynd™

As a first step the inventors have therefore assessed a tablet comprising the same amount of active ingredient as Slynd™ in a bioequivalence clinical trial to test whether the drug product chewed and swallowed had the same extent and rate of absorption. This clinical trial showed that the chewed test product had a similar AUC, but a higher Cₘₐₓ than the one observed for Slynd™. Therefore, test and reference products were not bioequivalent. Based on this outcome the inventors have developed a chewable drospirenone tablet with a reduced dose that meets the bioequivalence criteria for Cₘₐₓ and AUC as is further described in the Examples.

### Chewable Tablets of the Present Invention

According to the Guidance for industry: Quality Attribute Considerations for Chewable Tablets (U.S. Department of Health and Human Services, Food and Drug Administration, Center for Drug Evaluation and Research (CDER), August 2018) a variety of physical characteristics should be considered in the manufacturing process for chewable tablets. An ideal chewable tablet should be:
Easy to chew
Palatable (taste masked or of acceptable taste)
Of appropriate size and shape.
Able to disintegrate readily to facilitate dissolution

Critical quality attributes for chewable tablets should include hardness, disintegration, and dissolution, as well as all factors that may influence drug bioavailability and bioequivalence. No single quality characteristic is considered sufficient to control the performance of a chewable tablet. Instead, the goal should be to develop the proper combination of these attributes to ensure the performance of the chewable tablet for its intended use.

In order to provide a chewable oral contraceptive comprising drospirenone the inventors have departed from the approved Slynd™ tablet as reference product, which is a swallowable tablet. The reference product was compared with tablets comprising 4.0 mg of drospirenone, having the same composition as the reference product but with a mean hardness of 40 N (test product). The reference product was instructed to be swallowed, and the test product was instructed to be chewed before being swallowed by the test subjects. It was found that the chewing of the tablet leads to an acceptable extent of absorption (as assessed by AUC0-72) but a higher rate of absorption Cₘₐₓ of drospirenone when compared to the reference product.

Tablets comprising 4.0 mg of drospirenone, with an increased mean hardness of 40 N were found to not meet the bioequivalence criteria for Cₘₐₓ, see Table 2 in Example 1.

The inventors therefore studied possibilities to decrease the first oral absorption (Cₘₐₓ) that was presented in the bioequivalence study. Additionally, optimal balance for AUC and Cₘₐₓsimultaneously for bioequivalence compliance should be achieved.

As further detailed in Example 2 it was therefore assessed whether the dosage of drospirenone in the chewable tablet formulation could be reduced. The main criteria being that AUC and Cₘₐₓ stay within the acceptable ranges. As can be seen from the calculations in Example 2, the ranges between 3.4 to 3.7 mg of drospirenone are suitable, 3.5 mg being the most preferred amount.

As a further step, the effect of the hardness on the dissolution profile of the tablet was assessed. For this, different 4.0 mg chewable tablets with three different values for the hardness (25 N, 45 N, 65 N) were prepared and their dissolution profile tested against the reference product Slynd™ as described in Example 3. As a pre-step the in vitro dissolution media was optimized to account for the mechanical process of the chewing process and the different manufactured batches were analyzed using the optimized tool followed by in vitro dissolution testing (tested as crushed) as disclosed in Example 3.

Based on the in vivo and in vitro data obtained for the 4.0 mg chewable formulation a first IVIVC was developed and used to evaluate expected PK profiles for tablets with different hardness to select the most optimal combination of hardness and dose of the chewable tablet that would meet the bioequivalence criteria as compared to the Slynd™ formulation as disclosed in example 4.

As a result of this evaluation, the prototype with a hardness of 65 N and a dose of about 3.6 mg was expected to be the best candidate having PK parameters similar to the reference tablet. It was the one with the closest expected Cₘₐₓ ratio, and limited change on AUC ratio to the reference (Slynd™). As can be observed in FIG. 4A and B, the predictive error was for both AUC₀₋₇₂ₕ and Cₘₐₓ at a dose of 3.6 mg closer to the minimum of 0%. Additionally, a prototype with hardness between about 40 N-65 N at a dose of 3.5 mg was expected to be within the acceptance limits for both Cmax and AUC.

Based on the results from modelling IVIVC, 3.5 mg was the dose that would result into a bioequivalent drug product. For this reason, as a further step the effect of the hardness on the dissolution profile of a chewable tablet comprising 3.5 mg drospirenone was assessed. For this, different tablets comprising the composition as shown in table 4 of Example 2 with three different values for the hardness were prepared and their dissolution profile tested against the reference product Slynd™ as described in Example 5. As can be seen from FIG. 5 the prototype with a hardness of 65 N showed the most similar dissolution profile to Slynd™.

Based on IVIVC calculations, the final prototype selected for the new clinical study chewable of the chewable formulation had a hardness of 65 N and was administered at a dose of 3.5 mg.

A second clinical trial was performed in which the bioequivalence of a chewable oral tablet comprising a composition comprising drospirenone 3.5 mg was compared to the Slynd™ composition comprising 4.0 mg drospirenone, both tablets having a hardness of 65 N. As can be seen in table 6 of Example 7, chewable tablets comprising 3.5 mg of drospirenone with a hardness of 65 N meet the bioequivalence criteria for Cₘₐₓ and AUC. It can be further seen that increasing the hardness alone without lowering the dosage of drospirenone does not lead to a sufficient lowering of the Cₘₐₓ (see table 6 of Example 7).

An estimation of the optimal dose (balance between AUC₀₋₇₂ₕ and Cₘₐₓ) expected to be compliant for BE criteria was also derived for every hardness from the simulations using IVIVC (see FIG. 6). As can be seen the adjustment of the dosage together with the hardness can lead to the required BE meaning that a suitable chewable tablet can be obtained by combining the hardness of 15 N-100 N with the corresponding amount of drospirenone from 3.4 mg-3.9 mg.

To in more detail assess the role of the hardness for the dissolution profile, tablets with different hardness between 15 N to 100 N were prepared and their dissolution profile assessed (see Example 8). As can be seen from FIG. 7 the 3.5 mg chewable tablets comprising a hardness of 30 N-75 N show a similar dissolution profile to Slynd™, the most similar ones being tablets comprising a hardness of 40 N-65 N.

The present invention therefore relates in one aspect to an oral chewable tablet comprising a composition comprising 3.4 mg-3.7 mg, preferably 3.5 mg of drospirenone, wherein said tablet further has a hardness of 15 N to 100 N, preferably of 30 N-75 N, more preferably of 40 N-65 N and wherein the tablet does not comprise any estrogen.

In one embodiment the tablet comprises drospirenone as the only contraceptive ingredient.

In one embodiment the tablet comprises between 3.4 mg to 3.6 mg, between 3.4 mg to 3.5 mg, between 3.5 mg to 3.6 mg, between 3.5 mg to 3.7 mg or between 3.6 mg to 3.7 mg of drospirenone. The tablet of present invention can comprise 3.4 mg, 3.5 mg, 3.6 mg or 3.7 mg of drospirenone.

In a further embodiment, the tablet of the present invention has a hardness of 15 N to 100 N, 30 N to 75 N, 35 N to 65 N, 40 N to 60 N, 40 N to 65 N, 45 N to 55 N. The tablet of present invention can comprise a hardness of 15 N, 20 N, 30 N, 35 N, 40 N, 45 N, 50 N, 55 N, 60 N, 65 N, 70 N, 75 N, 80 N, 85 N, 90 N, 95 N, 100 N.

In one embodiment the oral chewable tablet comprises a composition comprising 3.8 mg or 3.9 mg of drospirenone and has a hardness of 85 N to 100 N, and wherein the tablet does not comprise any estrogen.

Estrogens commonly used for contraceptive purposes include, but are not limited to, estradiol, estradiol sulfamates, estradiol valerate, estradiol benzoate, ethinyl estradiol, estrone, estriol, estriol succinate, phytoestrogen and conjugated estrogens.

In some specific embodiments of the present invention, drospirenone may be combined with one or more progestogens. The term “progestogen”, as used herein, refers to any compound that binds and activates the progesterone receptor. Progestogens include, but are not limited to, 17-hydroxy progesterone esters, 19-nor-17-hydroxy progesterone esters, 17α-ethinyltestosterone and derivatives thereof, 17α-ethinyl-19-nor-testosterone and derivatives thereof, norethindrone, norethindrone acetate, ethynodiol diacetate, dydrogesterone, medroxy-progesterone acetate, norethynodrel, allylestrenol, lynoestrenol, fuingestanol acetate, medrogestone, norgestrienone, dimethiderome, ethisterone, cyproterone acetate, levonorgestrel, norgestrel, d-17α-acetoxy-13β-ethyl-17α-a-ethinyl-gon-4-en-3-one oxime, cyproterone acetate, gestodene, desogestrel, etonorgestrel, norgestimate, norelgestromin, chlormadinone and dienogest.

In some other embodiments, the drospirenone may be combined with one or more active ingredients which do not have contraceptive activities. Such active ingredients include, without being limited to, antiemetic agents, vitamins such as folic acid, Vitamin B12, Vitamin D, minerals and oligo elements such as iron, iodine, selenium and others. In a further embodiment the present invention relates to an oral chewable tablet comprising a composition comprising 3.4 mg-3.7 mg, preferably 3.5 mg of drospirenone, wherein said tablet further has a hardness of 15 N to 100 N, preferably from 30 N-75 N, more preferably of 40 N-65 N.

In a further embodiment the present invention relates to an oral chewable tablet comprising a composition comprising 3.4 mg-3.7 mg, preferably 3.5 mg of drospirenone as the only contraceptive ingredient, wherein said tablet further has a hardness of 15 N to 100 N, preferably from 30 N-75 N, more preferably of 40 N-65 N.

In one embodiment of the invention the chewable oral tablet as disclosed herein comprises or consists of 3.4 mg-3.7 mg, preferably 3.5 mg of drospirenone, and pharmaceutically acceptable excipients in the absence of a surfactant or disintegrant, wherein said tablet has a hardness of 30N-75N, more preferably of 40 N-65 N. Preferably, drospirenone is in non-micronized form. More preferably, the tablet does not comprise any estrogen.

In an even more preferred embodiment, the chewable oral tablet as disclosed herein comprises or consists of 3.4 mg-3.7 mg, preferably 3.5 mg of non-micronized drospirenone, and pharmaceutically acceptable excipients, wherein said excipients do not substantially modify the release of drospirenone, such as in the absence of a surfactant, a disintegrant or a polymeric or non-polymeric matrix, wherein said tablet has a hardness of 30N-75N, more preferably of 40 N-65 N. Preferably, wherein the tablet does not comprise any estrogen. Preferred embodiments of non-micronized drospirenone are as described herein.

In a further embodiment of the invention the chewable oral tablet as disclosed herein comprises or consists of 3.4 mg-3.7 mg, preferably 3.5 mg of drospirenone, and pharmaceutically acceptable excipients in the absence of a surfactant or disintegrant and wherein said tablet has a hardness of more than 75 N to 120 N, such as 100 N. Preferably, drospirenone is in micronized form. More preferably, the tablet does not comprise any estrogen.

In an even more preferred embodiment, the chewable oral tablet as disclosed herein comprises or consists of 3.4 mg-3.7 mg, preferably 3.5 mg of micronized drospirenone, and pharmaceutically acceptable excipients, wherein said excipients do not substantially modify the release of drospirenone, such as in the absence of a surfactant, a disintegrant or a polymeric or non-polymeric matrix, and wherein said tablet has a hardness of more than 75 N to 120 N, such as 100 N. Preferably, the tablet does not comprise any estrogen. Preferred embodiments of micronized drospirenone are as described herein.

In a further embodiment of the invention the chewable oral tablet as disclosed herein comprises or consists of 3.4 mg-3.7 mg, preferably 3.5 mg of drospirenone, and pharmaceutical excipients including a disintegrant and wherein said tablet has a hardness of more than 75 N to 120 N, such as 100 N. Preferably, drospirenone is in non-micronized form. More preferably, the tablet does not comprise any estrogen.

In an even more preferred embodiment, the chewable oral tablet as disclosed herein comprises or consists of 3.4 mg-3.7 mg, preferably 3.5 mg of non-micronized drospirenone, and pharmaceutical excipients, wherein said pharmaceutical excipients comprise a disintegrant and/or a surfactant and wherein said tablet has a hardness of more than 75 N to 120 N, such as 100 N. Preferably, the tablet does not comprise any estrogen. Preferred embodiments of non-micronized drospirenone are as described herein.

In one embodiment of the invention the chewable oral tablet as disclosed herein comprises or consists of 3.4 mg-3.7 mg, preferably 3.5 mg of drospirenone, and at least one pharmaceutically acceptable excipient and wherein said tablet has a hardness of 10 N to less than 30 N, such as 15 N. Preferably, drospirenone is in non-micronized form. More preferably, the tablet does not comprise any estrogen.

In an even more preferred embodiment, the chewable oral tablet as disclosed herein comprises or consists of 3.4 mg-3.7 mg, preferably 3.5 mg of non-micronized drospirenone, and one or more pharmaceutically acceptable excipients as described herein, wherein said pharmaceutical excipients comprise a polymeric or non-polymeric matrix, and wherein said tablet has a hardness of 10 N to less than 30 N, such as 15 N. Preferably, the tablet does not comprise any estrogen. Preferred embodiments of non-micronized drospirenone are as described herein.

The present invention further relates in another aspect to an oral chewable tablet comprising a composition comprising 3.4 mg-3.7 mg, preferably 3.5 mg of drospirenone, wherein the tablet has a dissolution profile characterized in that 20-40%, of the drospirenone initially present in said composition is dissolved within 30 minutes when the composition is subjected to an in vitro dissolution test in 900 ml of water with 0.6% polysorbate using USP apparatus 2 (paddles) at 100 rpm, preferably at a temperature of 37° C.±0.5° C., the percentages of drospirenone being related to the amount of drospirenone initially present in said tablet.

In one embodiment of the invention the chewable oral tablet as disclosed herein has a dissolution profile characterized in that no more than 30-40%, preferably no more than 35% of the drospirenone initially present in said composition is dissolved within 30 minutes and the tablet is subjected to an in vitro dissolution test in 900 ml of water with 0.6% polysorbate using USP apparatus 2 (paddles) at 100 rpm, preferably at a temperature of 37° C.±0.5° C., the percentages of drospirenone being related to the amount of drospirenone initially present in said composition.

In a preferred embodiment at least 75% of said drospirenone is dissolved after 4 hours.

In a preferred embodiment at least 50%, preferably at least 60%, 65% or 70% of said drospirenone is dissolved in a time range from about 3 hours to about 4 hours.

In one embodiment the tablet does not comprise any estrogen, preferably said tablet comprises drospirenone as the only contraceptive ingredient.

In a further embodiment the oral chewable tablet comprises drospirenone as the only contraceptive ingredient, preferably 3.4 mg-3.7 mg, preferably 3.5 mg of drospirenone, wherein said tablet further has a hardness of 15 N to 100 N, preferably of 30 N-75 N, more preferably of 40 N-65 N.

In one embodiment of the invention the chewable oral tablet as disclosed herein has a dissolution profile characterized in that no more than 40-60%, preferably no more than 40-50% of the drospirenone initially present in said composition is dissolved within 30 minutes and when the tablet is crushed, and the crushed tablet is subjected to an in vitro dissolution test in 900 ml of water with 0.6% polysorbate using USP apparatus 2 (paddles) at 75 rpm, preferably at a temperature of 37° C.±0.5° C., the percentages of drospirenone being related to the amount of drospirenone initially present in said composition.

In a preferred embodiment at least 75% of said drospirenone is dissolved after 4 hours.

In a preferred embodiment at least 50%, preferably at least 60%, 65% or 70% of said drospirenone is dissolved in a time range from about 3 hours to about 4 hours.

Chewable tablets in the prior art are usually immediate release tablets, as the chewing process leads to a tablet disintegration and quick release of the API. Delayed, modified or controlled release formulations are usually incompatible with chewing. The inventors have surprisingly found that the chewable oral tablets of present invention show a slow-release profile of drospirenone.

A third aspect of the invention relates to an oral chewable tablet comprising from 3.4 mg-3.7 mg, preferably 3.5 mg drospirenone, wherein the tablet is formulated such that the tablet provides a mean Cₘₐₓ of less than 30 ng/ml drospirenone, preferably of less than 25 ng/ml in a subject after a single administration of the oral dosage form when the tablet is chewed or disintegrated in the oral cavity of the subject prior to swallowing.

In a further embodiment the invention relates to an oral chewable tablet comprising from 3.4 mg-3.7 mg, preferably 3.5 mg drospirenone, wherein the tablet is formulated such that the tablet provides a mean Cₘₐₓ between 14 ng/ml to 22.5 ng/ml in a subject after a single administration of the oral dosage form when the tablet is chewed or disintegrated in the oral cavity of the subject prior to swallowing.

In a preferred embodiment at least 75% of said drospirenone is dissolved after 4 hours.

In a preferred embodiment at least 50%, preferably at least 60%, 65% or 70% of said drospirenone is dissolved in a time range from about 3 hours to about 4 hours.

In one embodiment the tablet does not comprise any estrogen, preferably said tablet comprises drospirenone as the only contraceptive ingredient.

In a further embodiment of this aspect the present invention relates to an oral chewable tablet comprising a composition comprising 3.4 mg-3.7 mg, preferably 3.5 mg of drospirenone, wherein said tablet further has a hardness of 15 N to 100 N, preferably from 30 N-75 N, more preferably of 40 N-65 N.

In a further embodiment of this aspect the present invention relates to an oral chewable tablet comprising a composition comprising 3.4 mg-3.7 mg, preferably 3.5 mg of drospirenone as the only contraceptive ingredient, wherein said tablet further has a hardness of 15 N to 100 N, preferably from 30 N-75 N, more preferably of 40 N-65 N.

A further feature of the chewable tablets described herein that can have an effect on patient compliance as well as the dissolution profile is its size. Size and shape of tablets and capsules affect the transit of the product through the pharynx and esophagus and may directly affect a patient's ability to swallow a particular drug product. Larger tablets and capsules have been shown to have a prolonged esophageal transit time. Smaller tablets generally have been shown to have significantly faster transit times. The inventors have therefore chosen to keep the chewable tablet at a relatively small size ensuring that it is on the hand not too small for chewing, but on the other hand not too large to swallow when crushed only partially during the chewing process.

In one embodiment of present invention the oral chewable tablet composition has a minimum diameter of between 4.0 to 6.0 mm, more preferred of 4.5 to 5.5 mm, most preferred of 5.0 mm. As shown in Example 10, this size is suitable to ensure an acceptable chewing difficulty index for the tablets. The inventors have tested various diameters and thicknesses of the tablets of present invention as can be seen in Example 10.

In a further embodiment the oral chewable tablet with a diameter of between 4.0 to 6.0 mm, more preferred of 4.5 to 5.5 mm, most preferred of 5.0 mm can have a thickness of between 1.9 to 2.9 mm, between 2.0 to 2.8 mm, between 2.1 to 2.8 mm, between 2.2 to 2.8 mm, between 2.3 to 2.8 mm, between 2.4 to 2.7 mm, 2.4 to 2.6 mm. In a preferred embodiment a tablet with a diameter of between 5 mm to 5.5 mm has a thickness between 2.2 mm to 2.9 mm, more preferred between 2.3 and 2.8 mm, even more preferred between 2.4 and 2.7 mm. Most preferred is a tablet with a diameter of 5 mm and a thickness of 2.4 mm.

In a further embodiment the oral chewable tablet has a surface area of between 50 to 85 mm², between 55 to 80 mm², between 60 to 75 mm², between 61 to 74 mm², between 62 to 73 mm², between 63 to 72 mm², between 64 to 71 mm². Most preferred is a tablet with a diameter of 5 mm and a surface area of between 64 to 65 mm², or a tablet with a diameter of 5.5 mm and a surface area of between 71 to 72 mm².

In a further embodiment the oral chewable tablet has a volume of between 29 to 55 mm³, between 31 to 52 mm³, between 33 to 54 mm³, between 35 to 50 mm³, between 36 to 47 mm³, between 37 to 45 mm³, between 38 to 43 mm³, between 39 to 41 mm³. Most preferred is a tablet with a diameter of 5 mm and a volume of between 39 and 40 mm³, or a tablet with a diameter of 5.5 mm and a volume of between 43 and 44 mm³.

One way to obtain the DRSP-containing composition of the invention is to use drospirenone in a particle form having an appropriate specific surface area. Drospirenone may be present in the pharmaceutical compositions of the invention in a non-micronized particle form. In a preferred embodiment drospirenone is present in a crystallized and non-micronized particle form, this can be obtained for instance by the method described in W02006061309.

Drospirenone in a particle form having a specific surface area from about 2000 cm²/g to about 8500 cm²/g may be suitable for obtaining the compositions of the invention. The specific surface area may be experimentally determined using the BET method (gas adsorption method). In some embodiments, the contraceptive composition of the invention comprises drospirenone in a particle form having a specific area from about 2000 cm²/g to about 8500 cm²/g.

Concerning the size particle distribution, drospirenone particles having a diameter greater than 200 μm shall be avoided in order to not drastically impair the in vitro dissolution rate and, thus, the in vivo bioavailability since such particles are poorly soluble.

Drospirenone may preferably have a d50 of less than 70 μm. In a preferred embodiment, the d50 of the drospirenone particles ranges from 10 μm to 60 μm. A d50 ranges from about 10 μm to about 60 μm encompasses a d50 of 10 μm, of 15 μm, of 20 μm, of 25 μm, of 30 μm, of 35 μm, of 40 μm, of 45 μm, of 50 μm, of 55 μm and of 60 μm.

In a further embodiment of the invention the drospirenone therefore has a d50 particle size ranging from 10 μm to 60 μm, preferably a d50 particle size ranging from 20 μm to 50 μm, most preferably from 10 μm to about 30 μm.

In some embodiments, the particle size distribution of the drospirenone present in the oral chewable tablet according to the invention is characterized by:
(i) a d90 particle size less than about 100 μm, and/or
(ii) a d50 particle size ranging from about 10 μm to about 60 μm and/or
(iii) a d10 particle size more than about 3 μm.

In some other embodiments, the d50 of drospirenone particles ranges from about 10 μm to about 30 μm. In such embodiments, the particle size distribution of the drospirenone present in the oral chewable tablet according to the invention is characterized by at least one of the following characteristics:
(i) a d90 particle size less than about 100 μm, and/or
(ii) a d10 particle size more than about 3 μm.

As used herein, the term “about” before a “specific value” defines a range from “the specific value minus 10% of the specific value” to “the specific value plus 10% of the specific value”. For example, “about 50” defines a range from 45 to 55.

As used herein, by “d90 particle size” it is meant that the particle size distribution is such that 90% of the particles have a particle size diameter of less than the specified value.

As used herein, by “d50 particle size” it is meant that the particle size distribution is such that 50% of the particles have a particle size diameter of less than the specified value.

As used herein, by “d10 particle size” it is meant that the particle size distribution is such that 10% of the particles have a particle size diameter of less than the specified value.

The drospirenone particle size distribution, in particular d90, d10 and d50 values, may be determined by well-known methods of the prior art such as sieve analysis, laser diffraction methods, photoanalysis or optical counting methods. Laser diffraction methods are particularly preferred, for example with water as solvent to disperse the particles using a Helos Particle size analysis (SympaTec).

d90 particle size less than about 100 μm include d90 particle sizes less than about 90 μm, 80 μm, 70 μm, 60 μm, 55 μm, 50 μm, 45 μm, 40 μm, 38 μm, 36 μm, 34 μm, 32 μm, 30 μm, 28 μm, 26 μm, 24 μm, 22 μm, 20 μm.

d50 particle size values ranging from about 10 μm to about 30 μm include values of about 10μm, 11 μm, 12 μm, 13 μm, 14 μm, 15 μm, 16 μm, 18 μm, 19 μm, 20 μm, 21 μm, 22 μm, 23μm, 24 μm, 25 μm, 26 μm, 27 μm, 28 μm, 29 μm, 30 μm.

d10 particle size values more than about 3 μm include d10 particle size values more than about 3 μm, 3.5 μm 4.5 μm, 5 μm, 6 μm, 7 μm, 8 μm, 9 μm, 10 μm, 11 μm, 12 μm.

It goes without saying that d10 particle size value is smaller than d50 particle size value which is smaller than d90 particle value.

In some embodiments, the oral chewable tablet of the invention comprises drospirenone in a particle form having a particle size distribution having a combination of two characteristics selected from:
(i) a d90 particle size less than about 100 μm,
(ii) a d50 particle size ranging from about 10 μm to about 30 μm and
(iii) a d10 particle size more than about 3 μm.

In other words, the particle size distribution of DRSP display a combination of characteristics selected from characteristic (i) and characteristic (ii), characteristic (i) and characteristic (iii), and, characteristic (ii) and characteristic (iii).

In some other embodiments, the oral chewable tablet of the invention comprising drospirenone in a non-micronized form having a particle size distribution characterized in that:
(i) d90 particle size is less than about 100 μm,
(ii) d50 particle size ranging from about 10 μm to about 30 μm and
(iii) d10 particle size is more than about 3 μm.

In a preferred embodiment, the DRSP particle distribution is further characterized in that d90 particle size value is less than 50 μm and in that no particle has a size greater than 80 μm.

In some embodiments, the oral chewable tablet of the invention comprises drospirenone in a particle form having a d90 particle size which ranges from about 20 μm to about 40 μm, a d50 particle size which ranges from about 10 μm to about 30 μm and a d10 which ranges from about 3 μm to about 9 μm and wherein no particle has a size greater than 80 μm, more preferably no particle has a size greater than 60 μm.

In some other embodiments, the oral chewable tablet of the invention comprises drospirenone in a particle form having
(i) a d90 particle size which ranges from about 30 μm to about 40 μm,
(ii) a d50 particle size which ranges from about 15 μm to about 25 μm and
(iii) a d10 which ranges from about 5 μm to about 9 μm and wherein no particle has a size greater than 80 μm, more preferably no particle has a size greater than 60 μm.

In some other embodiments, the oral chewable tablet of the invention comprises drospirenone in a particle form having a specific surface area from about 2000 cm²/g to about 8000 cm²/g and having a d50 particle size ranges from 10 μm to 60 μm.

In some other embodiments, the oral chewable tablet of the invention may comprise drospirenone in micronized form.

In some other embodiments, the oral chewable tablet of the invention comprises drospirenone in a micronized form having a particle size distribution characterized in that d90 particle size is less than about 10 μm.

Drospirenone in a particle form having a specific surface area higher than 8500 cm²/g may be suitable for obtaining the compositions of the invention. In some embodiments, the contraceptive composition of the invention comprises drospirenone in a particle form having a specific area from about 9000 cm²/g to 12000 cm²/g.

### Uses and Methods

The present invention further relates to the use of the oral chewable tablet as described herein for providing contraception to an individual.

In one embodiment the present invention relates to the use of the oral chewable tablet as described herein for providing contraception to an individual, preferably comprising administering one tablet once daily to said individual over a period of 24 to 28 days.

When used for contraceptive purposes, the oral chewable tablet is administered to a female subject of child-bearing age i.e., from the puberty to the menopause. Women of child-bearing age also include women in perimenopause.

The present invention furthermore relates to a method for obtaining a mean Cₘₐₓ of less than 30 ng/ml drospirenone in a subject, preferably of less than 25 ng/ml the method comprising administering to the subject a single oral dosage form comprising from 3.4 mg-3.7 mg, preferably 3.5 mg drospirenone, wherein the oral dosage form is chewed or disintegrated in the oral cavity of the subject prior to swallowing.

In one embodiment of the method the oral dosage form does not comprise any estrogen, preferably drospirenone is the only active ingredient of the oral dosage form.

In a preferred embodiment of the method the oral dosage form is a chewable oral tablet as disclosed herein above.

The present invention furthermore relates to a method for providing contraception in a subject, comprising administering to the subject an oral dosage form comprising from 3.4 mg-3.7 mg, preferably 3.5 mg drospirenone, wherein the oral dosage form is chewed or disintegrated in the oral cavity of the subject prior to swallowing, and wherein the administering results in a mean Cₘₐₓ of less than 30 ng/ml drospirenone, preferably of less than 25 ng/ml in the subject after a single administration of the oral dosage form.

In a more general aspect, the present invention provides a pharmaceutical DRSP-containing composition characterized in that, when orally administered, a single daily dosage unit of said composition is adapted to provide a pharmacokinetic profile for DRSP having a mean Cₘₐₓ which is less than 25 ng/ml in a subject after a single administration of the oral dosage form.

The said pharmaceutical DRSP-containing composition may be further characterized in that, when orally administered, a single daily dosage unit of said composition is adapted to provide a pharmacokinetic profile for DRSP having a mean tₘₐₓ which is at least about 1 h. It goes without saying that the administration of a single dosage unit of the said composition provides a mean AUC₀ₕ₋ₜₗₐₛₜ which is sufficient to produce the pharmaceutical or the biological effect which is sought by the administration of drospirenone.

When the composition of the invention is used as a contraceptive, it may be further required that the mean AUC₀ₕ₋ₜₗₐₛₜ obtained upon the administration of a single daily dosage unit of said composition is at least about 300 ng*ml/h. In other words, in some embodiments, the daily dosage unit of the pharmaceutical composition according to the invention possesses a combination of physical and/or chemical features such that, when orally administered, the said daily dosage unit is adapted to provide a pharmacokinetic profile in a subject having the following characteristics:
(i) a mean Cₘₐₓ which is less than about 30 ng/ml
(ii) a mean tₘₐₓ of at least about 1 h
and, optionally, a mean AUC₀ₕ₋ₜₗₐₛₜ of at least about 300 ng*h/ml
after a single administration of the oral dosage form

In some embodiments, the pharmaceutical composition of the invention displays all the previous mentioned pharmacokinetic characteristics.

A mean AUC₀ₕ₋ₜₗₐₛₜ of at least about 300 ng*h/mL includes a mean AUC₀ₕ₋ₜₗₐₛₜ of at least about 310 ng*h/mL, at least about 320 ng*h/mL, at least about 330 ng*h/mL, at least about 340 ng*h/mL, at least about 350 ng*h/mL, at least about 360 ng*h/mL, at least about 370 ng*h/mL, at least about 380 ng*h/mL, at least about 390 ng*h/mL, at least about 400 ng*h/mL, at least about 410 ng*h/mL at least about 420 ng*h/mL, at least about 430 ng*h/mL.

In some embodiments, the mean AUC₀ₕ₋ₜₗₐₛₜ is at least about 350 ng*h/ml.

A mean tₘₐₓ of at least about 1 h includes a mean tₘₐₓ of at least about 1.5 h, of at least 2.0 h, of at least about 2.5 h, of at least 2.0 h. In a preferred embodiment the mean tₘₐₓ does not exceed 4 hours in order to not significantly impair the bioavailability of DRSP. Thus, the mean tₘₐₓ preferably ranges from 1 h to 4 h.

In some embodiments, a tₘₐₓ ranging from 2.0 h to 3.0 h is preferred.

A mean Cₘₐₓ which is less than about 30 ng/ml includes a Cₘₐₓ less than about 27 ng/ml, less than about 25 ng/ml, less than about 24 ng/ml, less than about 22 ng/ml, less than about 20 ng/ml, less than about 19 ng/ml, less than about 18 ng/ml, less than about 17 ng/ml, less than about 16 ng/ml, less than about 15 ng/ml, less than about 14 ng/ml.

In some embodiments, the mean Cₘₐₓ ranges from 14 ng/ml to 30 ng/ml. In other embodiments, the mean Cₘₐₓ ranges from 15 ng/ml to 25 ng/ml, more preferably from 14 ng/ml to 22 ng/ml

In certain embodiments, the daily dosage unit of the pharmaceutical composition according to the invention is adapted to provide a pharmacokinetic profile having the following characteristics:
(i) a mean Cₘₐₓ ranges from 14 ng/ml to 25 ng/ml,
(ii) a mean tₘₐₓ ranges from 1 h to 4 h, and
(iii) optionally, a mean AUC₀ₕ₋ₜₗₐₛₜ of at least about 300 ng*h/ml, when the said daily dosage unit is administered under fasting condition.

In a preferred embodiment, the daily dosage unit of the pharmaceutical composition according to the invention is adapted to provide a pharmacokinetic profile having the following characteristics:
(i) a mean Cₘₐₓ ranges from 14 ng/ml to 22 ng/ml, more preferably from 14 ng/ml to 22 ng/ml
(ii) a mean tₘₐₓ ranges from 1.5 h to 3 h, and
(iii) optionally, a mean AUC₀ₕ₋ₜₗₐₛₜ of at least about 300 ng*h/ml, when the said daily dosage unit is administered under fasting condition

In a preferred embodiment, the pharmacokinetic parameters (namely Cₘₐₓ, tₘₐₓ and AUC₀ₕ₋ₜₗₐₛₜ) are determined after the first oral administration of a single unit dosage

In a preferred embodiment the oral dosage form does not comprise any estrogen. In an even more preferred embodiment drospirenone is the only active ingredient of the oral dosage form.

### Kit

The present invention furthermore relates to a kit, preferably a contraceptive kit, comprising one or more packaging units, wherein each packaging unit comprises from 24 to 28 active daily dosage units, each daily dosage unit comprising an oral chewable tablet as described above.

In a preferred embodiment, the contraceptive kit comprises one or more packaging units wherein each packaging unit comprises 24 to 28 daily active dosage units, each dosage unit comprising an oral chewable tablet comprising 3.4 mg-3.7 mg, preferably 3.5 mg of drospirenone, wherein said tablet further has a hardness of 15 N-100 N, preferably 30N-75N, more preferably of 40 N-65 N.

In a preferred embodiment, the contraceptive kit comprises one or more packaging units wherein each packaging unit comprises 24 to 28 daily active dosage units, each dosage unit comprising an oral chewable tablet comprising 3.4 mg-3.7 mg, preferably 3.5 mg of drospirenone, wherein the tablet has a dissolution profile characterized in that 20-40%, of the drospirenone initially present in said composition is dissolved within 30 minutes when the tablet is subjected to an in vitro dissolution test in 900 ml of water with 0.6% polysorbate, using USP apparatus 2 (paddles) at 100 rpm, preferably at a temperature of 37° C.±0.5° C., the percentages of drospirenone being related to the amount of drospirenone initially present in said tablet.

In one embodiment of the invention the contraceptive kit comprises one or more packaging units wherein each packaging unit comprises 24 to 28 daily active dosage units, each dosage unit comprising an oral chewable tablet comprising 3.4 mg-3.7 mg, preferably 3.5 mg of drospirenone, wherein the tablet has a dissolution profile characterized in that no more than 30-40%, preferably no more than 35% of the drospirenone initially present in said composition is dissolved within 30 minutes and when the tablet is crushed, and the crushed tablet is subjected to an in vitro dissolution test in 900 mL of water with 0.6% polysorbate using USP apparatus 2 (paddles) at 100 rpm, preferably at a temperature of 37° C.±0.5° C., the percentages of drospirenone being related to the amount of drospirenone initially present in said composition.

In one embodiment of the invention the contraceptive kit comprises one or more packaging units wherein each packaging unit comprises 24 to 28 daily active dosage units, each dosage unit comprising an oral chewable tablet comprising 3.4 mg-3.7 mg, preferably 3.5 mg of drospirenone, wherein the tablet has a dissolution profile characterized in that no more than 40-60%, preferably no more 40-50% of the drospirenone initially present in said composition is dissolved within 30 minutes when the tablet is crushed, and the crushed tablet is subjected to an in vitro dissolution test in 900 ml of water with 0.6% polysorbate, using USP apparatus 2 (paddles) at 75 rpm, preferably at a temperature of 37° C.±0.5° C., the percentages of drospirenone being related to the amount of drospirenone initially present in said tablet.

In a preferred embodiment at least 75% of said drospirenone is dissolved after 4 hours.

In a preferred embodiment at least 50%, preferably at least 60%, 65% or 70% of said drospirenone is dissolved in a time range from about 3 hours to about 4 hours.

In one embodiment of the kit of the present invention the 24 to 28 active daily dosage units do not comprise an estrogen. Estrogens commonly used for contraceptive purposes include, but are not limited to, estradiol, estradiol sulfamates, estradiol valerate, estradiol benzoate, ethinyl estradiol, estrone, estriol, estriol succinate, phytoestrogen and conjugated estrogens.

In a preferred embodiment of the present invention drospirenone is the only contraceptive active ingredient in the 24 to 28 active daily dosage units.

Each packaging unit optionally comprises from 1 to 4 daily dosage units of a pharmaceutically acceptable placebo.

In a preferred embodiment the contraceptive kit comprises one or more packaging units wherein each packaging unit comprises 24 daily active dosage units and 4 placebo dosage units, each dosage unit comprising an oral chewable tablet comprising 3.4 mg-3.7 mg, preferably 3.5 mg of drospirenone, wherein the tablet has a dissolution profile characterized in that 20-40%, of the drospirenone initially present in said composition is dissolved within 30 minutes when the tablet is subjected to an in vitro dissolution test in 900 ml of water with 0.6% polysorbate, using USP apparatus 2 (paddles) at 100 rpm, preferably at a temperature of 37° C.±0.5° C., the percentages of drospirenone being related to the amount of drospirenone initially present in said tablet.

In some embodiments, the kit is characterized in that each packaging unit comprises 28 daily active dosage units according to the invention. Such a kit is particularly appropriate to perform a contraceptive method which consists in administering “continuously” the active daily dosage units of the invention without any placebo phase in which no contraceptive is given.

In some other embodiments, the kit is characterized in that the daily dosage unit is administered in accordance with a so-called flexible administration regimen. In this case, the daily dosage unit is administered for at least 24 days and at most for 116 days. A decision about the duration of the administration phase is left to be made by the user within the said limits, that is to say, the user herself can decide whether to initiate a second phase wherein no contraceptive composition is administered to the female patient over a period of 1 to 4 consecutive days.

The said kit comprises one or more packaging units. One or more packaging units includes, without being limited to, 1 packaging unit, 2 packaging units, 3 packaging units, 4 packaging units, 5 packaging units and 6 packaging units.

The packaging unit as described above may have one of the conventional forms usually used for oral contraceptives. For example, the packaging unit may be a conventional blister pack comprising the appropriate number of dosage units in a sealed blister pack (e.g. an aluminium blister) with a cardboard, paperboard, foil or plastic backing and enclosed in a suitable cover. Each blister container may be conveniently numbered or marked in order to facilitate compliance. The packaging unit may contain daily dosage units in the order in which they are to be taken.

The kit of the invention may comprise any of the pharmaceutical compositions as disclosed herein after.

The kit of the invention may further comprise other appropriate components such as instructions for use.

### Pharmaceutical Compositions

In a further embodiment of the present invention the oral chewable tablet further comprises one or more pharmaceutically acceptable excipients.

The pharmaceutically acceptable excipients of the oral chewable tablet of the present invention include, but are not limited to, binders, fillers, glidants, lubricants, granulating aids, colorants, anti-caking agents, plasticizers, disintegrants dyes, antioxidants, anti-adherents, softeners, preservatives and flavorants that are conventional in the pharmaceutical art.

Exemplary formulations have been prepared and their dissolution profile tested as shown in Example 11. All formulations show the desired dissolution profile.

The suitable binders of the oral chewable tablet of present invention include, but are not limited to, microcrystalline cellulose, hydroxypropyl cellulose, hydroxypropyl methyl cellulose, hydroxyethyl cellulose, methylcellulose, polyvinylpyrrolidone (povidone), sodium carboxymethyl cellulose, calcium carboxymethyl cellulose, gums, such as gum tragacanth, acacia gum and gelatin and/or mixtures thereof. Preferred binders are methylcellulose, povidone, gelatin and microcrystalline cellulose. Especially preferable is microcrystalline cellulose. Binders may be present in an amount from about 30% to about 85% by weight, from about 35% to about 80%, from about 40% to about 75%, from about 45% to about 70% preferably from about 50% to about 65% by weight, and more preferably from about 55% to about 60% by weight, preferably about 56%, about 57%, about 58%, about 59% by weight of the total weight of the composition.

In one embodiment binders may also be present in an amount from about 0.5% to about 20% by weight, preferably from 1% to 10% by weight, and more preferably from 2 to 7% by weight, preferably about 5% by weight of the total weight of the composition.

Suitable fillers, also known as diluents, include, but are not limited to, starch, corn starch, pregelatinized starch, modified starch, powdered cellulose, silicified cellulose, lactose monohydrate, anhydrous lactose, mannitol, sorbitol, sucrose, fructose, dextrose, dibasic calcium phosphate dihydrate, calcium sulfate trihydrate, calcium sulfate dihydrate, calcium carbonate and/or mixtures thereof. Preferred fillers are starch, starch modified with dodecanoyl chloride (starch DC), mannitol, calcium carbonate and sorbitol, and anhydrous lactose. Especially preferable is anhydrous lactose. Fillers may be present in an amount from about 10% to about 95% by weight, from about 11% to about 90%, from about 12% to about 80%, from about 13% to about 70%, from about 14% to about 65%, from about 15% to about 60%, from about 16% to about 55%, from about 17% to about 50%, from about 18% to about 45%, from about 19% to about 40%, from about 20% to about 35%, preferably from about 25% to about 35% by weight of the total weight of the composition. In one embodiment the fillers are present in an amount of about 26%, about 27%, about 28%, about 29%, about 30%, about 31%, about 32%, about 33%, about 34% by weight of the total weight of the composition.

Fillers may be presents in an amount from about 20% to about 95% by weight, preferably from 30% to 90% by weight, and more preferably from 35% to 80% by weight, even more preferably from 30% to 60%, including about 40%, about 45%, about 50%, about 55% and about 60% by weight of the total weight of the composition.

Lubricants suitable for present invention include, but are not limited to, talc, alkaline earth salts of stearic acid, such as magnesium stearate and calcium stearate, stearic acid, glycerin palmitostearate, stearyl fumarate, zinc stearate, propylene glycol, PEG, PEG 6000, vegetable oil, sodium benzoate, sodium lauryl sulfate, sodium dodecyl sulfate, magnesium lauryl sulfate, mineral oil polyoxyethylene monostearate and/or mixtures thereof. Preferred lubricants are stearic acid, PEG 6000, sodium dodecyl sulfate and magnesium stearate. In preferred embodiments, the lubricant is magnesium stearate. The lubricant may be present in an amount from about 0% to 5%, from about 0.05% to about 4%, from about 0.1% to about 3% by weight, preferably from about 0.1% to about 2.5%, preferably about 0.3%, about 0.4%, about 0.5%, about 0.6%, about 0.7%, about 0.8%, about 0.9%, about 1%, about 1.1%, about 1.2%, about 1.3%, about 1.4%, about 1.5%, about 1.6%, about 1.7%, about 1.8%, about 1.9%, about 2% by weight of the total weight of the composition.

Examples of glidants include silicon dioxide, magnesium trisilicate, powdered cellulose, starch, talc and tribasic calcium phosphate and/or mixtures thereof. In preferred embodiments, the glidant is silicon dioxide. The glidant may be present in an amount from about 0% to 5%, from about 0.05% to about 4%, from about 0.1% to about 3% by weight, preferably from about 0.1% to about 2.5%, preferably about 0.3%, about 0.4%, about 0.5%, about 0.6%, about 0.7%, about 0.8%, about 0.9%, about 1% by weight of the total weight of the composition.

Pharmaceutically acceptable excipients that may be used are, in particular, described in the Handbook of Pharmaceuticals Excipients, American Pharmaceutical Association (Pharmaceutical Press; 9th Revised edition, October 2020).

In some embodiments, the oral chewable tablet of the invention comprises at least one or more excipients selected from the group of binders, fillers, glidants and lubricants.

In one embodiment of the oral chewable tablet of present invention said pharmaceutically acceptable excipients are at least one binder and at least one filler, and wherein:
(i) the amount of drospirenone accounts for 1% to 10% by weight
(ii) the amount of the at least one binder accounts for 50% to 65% by weight and
(iii) the amount of the at least one filler accounts for 25% to 35% by weight,
   the percentages by weight being related to the total weight of the said tablet.

In a further embodiment of the present invention, the oral chewable tablet further comprises at least one glidant and at least one lubricant wherein:
(i) the amount of the at least one glidant accounts for 0.2% to 6% by weight and
(ii) the amount of the at least one lubricant accounts for 0.2% to 2.5% by weight the percentages by weight being related to the total weight of the said tablet.

In a preferred embodiment of the oral chewable tablet of the present invention
(i) the at least one binder is microcrystalline cellulose,
(ii) the at least one filler is anhydrous lactose,
(iii) the at least one glidant is silicon dioxide, and
(iv) the at least one lubricant is magnesium stearate.

The dosage form according to the invention can also comprise a disintegration agent. Disintegrating agents may be selected from the group consisting of low-substituted hydroxypropyl cellulose, sodium carboxymethyl cellulose, calcium carboxymethyl cellulose, crospovidone, sodium croscarmellose, and/or mixtures thereof. Preferred disintegrants are crospovidone and croscarmellose. Disintegrating agents may be present in an amount from about 0% to about 50% by weight, preferably from about 5% to about 45% by weight, and more preferably from 10% to 40% by weight of the total weight of the tablet. In one embodiment the disintegrant, preferably crospovidone, is present in an amount from about 0.1% to about 2.5%, preferably from about 0.5% to about 2.0% by weight of the total weight of the tablet.

The dosage form according to the invention can also comprise a retarding agent, such as for example HPMC (Methocel K100), in an amount from about 5% to about 15%, from about 8% to about 12%, preferably from about 9% to about 11% by weight of the total weight of the tablet.

In some embodiments of the invention DRSP is dispersed in a matrix. In some embodiments of the invention DRSP in the form of multiparticulates.

In one embodiment of the above aspect, the pharmaceutically acceptable matrix is a polymeric matrix, a non-polymeric matrix, or a combination thereof.

The polymeric matrix includes, but is not limited to, hydroxypropylmethyl cellulose; hydroxypropyl cellulose; hydroxyethyl cellulose; hydroxymethyl cellulose; carboxymethyl cellulose; sodium carboxymethyl cellulose; carboxymethylcellulose calcium; polyvinyl pyrrolidone; polyethylene oxide; polyvinyl alcohol; methyl cellulose; ethyl cellulose; propyl cellulose; ethylmethyl cellulose; isopropyl cellulose; ethylpropyl cellulose; butyl cellulose; benzyl cellulose; cellulose esters such as cellulose acetate, cellulose butyrate, cellulose propionate, cellulose butyrate, and cellulose acetate propionate; cellulose cyanoalkyl ethers such as cyanoethyl cellulose, cyanomethyl cellulose, cyanoethylmethyl cellulose, and cyanopropyl cellulose; methacrylic acid-acrylic acid copolymers (e.g., Eudragit RS, Eudragit RL, Eudragit NE, Eudragit RS PO, and Eudragit RL PO); methacrylic acid copolymers; hydroxypropyl methylcellulose phthalate; hydroxypropyl methylcellulose acetate succinate; cellulose acetate phthalate; and mixtures thereof.

The non-polymeric matrix includes, but is not limited to, sugar and sugar alcohols for example sucrose, lactose, fructose, maltose, raffinose, sorbitol, lactitol, mannitol, maltitol, erythritol, threitol, adonitol, arabitol, xylitol, dulcitol, inositol, trehalose, isomalt, 30 inulin, and maltodextrin; cyclodextrin, for example 13-cyclodextrin and hydroxypropyl-13-cyclodextrin; polyethylene glycol; polyethylene glycol esters; medium chain triglycerides; fatty acids; fatty alcohols; waxes; fatty acid esters and mixtures thereof.

In some particularly preferred embodiments, the dosage form of the invention is coated to prevent substantial direct contact of DRSP with the oral cavity (e.g. tongue, oral mucosa), oropharyngeal mucosal surface, oesophagus or stomach. In some preferred embodiments, the dosage form of the invention comprises an oral formulation which is coated with a film or polymer.

In a preferred embodiment, the tablet according to the invention does not comprise a significant amount of surfactant agent. A significant amount of a surfactant agent may impair the in vitro dissolution profile of DRSP by increasing its initial rate of dissolution. Suitable surfactant agents may be selected from the group consisting of ionic surfactants, such as sodium lauryl sulfate, phospholipids, glycerol monooleate, docusate sodium, or non-ionic surfactant, polyoxyethylene sorbitan fatty acid esters such as polysorbate 80, polyoxyethylene stearates, poloxamer, polyoxyethylene alkyl ethers.

If present, the surfactant is preferably in an amount of from about 0.01 weight percent (wt %) to about 5 wt %, more preferably in an amount of from about 0.1 wt % to about 1 wt %, based on the total weight of the tablet. In a most preferred embodiment, the tablet does not contain a surfactant agent.

The tablet as described herein may be suitable for administration as the daily active oral form in various administration regimens, preferred administration regimens are described herein above for contraceptive and also for medical purposes referred to herein.

It is contemplated that any features described herein can optionally be combined with any of the embodiments of any medical or contraceptive use, composition, kit, contraceptive methods, methods of treatment, or method of manufacturing of the invention; and any embodiment discussed in this specification can be implemented with respect to any of these. It will be understood that particular embodiments described herein are shown by way of illustration and not as limitations of the invention.

All publications and patent applications are herein incorporated by reference to the same extent as if each individual publication or patent application was specifically and individually indicated to be incorporated by reference.

The use of the word “a” or “an” may mean “one,” but it is also consistent with the meaning of “one or more,” “at least one,” and “one or more than one”. The use of the term “another” may also refer to one or more. The use of the term “or” in the claims is used to mean “and/or” unless explicitly indicated to refer to alternatives only or the alternatives are mutually exclusive.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. The term “comprises” also encompasses and expressly discloses the terms “consists of” and “consists essentially of”. As used herein, the phrase “consisting essentially of” limits the scope of a claim to the specified materials or steps and those that do not materially affect the basic and novel characteristic(s) of the claimed invention. As used herein, the phrase “consisting of” excludes any element, step, or ingredient not specified in the claim except for, e.g., impurities ordinarily associated with the element or limitation.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “around”, “approximately” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by±1, 2, 3, 4, 5, 6, 7, 8, 9, or 10%. Accordingly, the term “about” may mean the indicated value±5% of its value, preferably the indicated value±2% of its value, most preferably the term “about” means exactly the indicated value (±0%).

The following examples serve to illustrate the present invention and should not be construed as limiting the scope thereof.

### EXAMPLES

Example 1: Bioequivalence (BE) Study 1: Cmax of 4.0 Mg Drospirenone Chewable is Not BE

The primary objective of the first study was to determine the effect on bioavailability of a Drospirenone 4.0 mg tablet using different modes of administration: chewed and swallowed without water (Test1) and chewed and swallowed with water (240 mL) (Test 2) vs the reference Slynd™ 4 mg tablets (Reference RLD) swallowed with water.

The study was performed in 48 female volunteers in each test. In test 1 only 42 volunteers finished the study and in test 2, 46 volunteers.
Test products: Drospirenone 4.0 mg Chewable Tablets.
Reference product: Slynd™ (drospirenone) 4.0 mg tablets.

The formula for both products is the same and as depicted in table 1 here below.

TABLE 1
Composition of tablets Slynd ™ (drospirenone) 4.0
mg tablets and Drospirenone 4.0 mg Chewable Tablets

| Material | mg/tablet |
|---|---|
| Drospirenone | 4.00 |
| Microcrystalline cellulose | 33.02 |
| Anhydrous lactose | 17.50 |
| Silicon dioxide | 0.29 |
| Magnesium stearate | 0.29 |
| Coating (Opadry II 85F18422 white) | 1.65 |
| TOTAL | 56.75 |

The manufacturing process in both cases is a direct blend followed by the tableting and the addition of an aesthetical coating with the same coating material.

Drospirenone was prepared according to a process similar to the one described in W02006/061309. In order to obtain non-micronized DRSP with an appropriate particle size distribution, DRSP was subjected to an additional process of precipitation as described in WO2012/000981. Particle size distribution is as described in Example 1 of WO2012/000981, drospirenone particle size being characterized by a d50 between 20 and 50 μm.

The results obtained of the BE study 1 were as described in Table 2 below:

TABLE 2

| | Test-1 | Test-2 | Reference |
|---|---|---|---|
| Parameter (unit) | (n = 42) | (n = 46) | (n = 46) |
| Cₘₐₓ (ng/mL) | 25.47 (25.9%) | 25.06 (23.8%) | 17.80 (33.4%) |
| Tₘₐₓ (h) | 2.00 (1.00-4.00) | 2.00 (1.00-4.00) | 3.00 (1.75-6.00) |
| AUC₀₋₇₂ (ng · h/mL) | 476.28 (23.9%) | 449.57 (21.1%) | 428.59 (24.6%) |
| λ₂ (1/h) | 0.020 (22.4%) | 0.021 (24.6%) | 0.020 (24.0%) |
| t_{1/2} (h) | 36.70 (25.7%) | 34.98 (28.5%) | 36.56 (28.7%) |

n—Number of Subjects
Tₘₐₓ values are median with range between parenthesis

The results showed similarity on extent of absorption (AUCₒ₋₇₂ₕ), having a ratio of approximately 100%, but higher rate of absorption (Cₘₐₓ), having a ratio of approximately 145%, between the reference product (Slynd™ drospirenone 4 mg) and the test product (Drospirenone 4 mg chewable tablets) taken chewed and swallowed, with and without water.

Based on these results, and as can be seen in FIG. 1 Test 1 (T1) and Test 2 (T2) curves are superimposable and of greater rate of absorption than the Reference (R) (Cₘₐₓ and Tₘₐₓ). The greater rate of absorption after chewing administration is unlikely to result from buccal absorption due to very short oromucosal exposure and similarity of profiles of with and without water administration (T1 and T2).

In this Clinical study the palatable properties of the drug product were also included. Subjects sensory experience was in general neutral (value of 3) and slightly better after administration with water.

### Conclusions

The higher rate of absorption after chewing administration is considered unlikely to result from buccal absorption due to the very short oromucosal exposure
similarity of profiles of the “with” and “without” water administration (T1 and T2)

### Example 2: Dose reduction

In order to try to decrease the Cmax that was found in the BE study 1 for the chewable drospirenone tablets, pharmacokinetic (PK) calculations were conducted with reduced dosages of drospirenone, starting from a dose of 4.0 mg.

TABLE 3
Preliminary calculations for Drospirenone dosage

| | Test 1 | | Test 2 | | | | | |
|---|---|---|---|---|---|---|---|---|
| Simulated | (Without water) | | (With water) | | Ratio T1/R | | Ratio T2/R | |
| dose (mg) | Cₘₐₓ | AUC_{0-72 h} | Cₘₐₓ | AUC_{0-72 h} | Cₘₐₓ | AUC_{0-72 h} | Cₘₐₓ | AUC_{0-72 h} |
| 3.4 | 20.95 | 401.22 | 20.27 | 377.66 | 108.72 | 87.10 | 105.19 | 81.98 |
| 3.5 | 21.57 | 413.02 | 20.87 | 388.76 | 111.94 | 89.66 | 108.30 | 84.39 |
| 3.6 | 22.18 | 424.82 | 21.46 | 399.87 | 115.13 | 92.22 | 111.36 | 86.81 |
| 3.7 | 22.80 | 436.62 | 22.06 | 410.98 | 118.33 | 94.78 | 114.48 | 89.22 |
| 4 | 24.65 | 472.022 | 23.85 | 444.30 | 127.92 | 102.47 | 123.77 | 96.45 |

The goal was to find formulations which are bioequivalent both in terms of AUC and Cₘₐₓ to Slynd™ 4 mg tablets. For this purpose, a parameter was considered bioequivalent when the ratio between the test and reference product for this parameter is found between 90% and 110%. In a first step a simulation of PK profiles of a range of lower doses of the chewable tablets were modelled assuming a linear PK behavior (that was indeed confirmed afterwards) and AUC₀₋₇₂ₕ and Cₘₐₓ of simulated profiles (the range of tested doses was 3.0 mg to 4.0 mg) compared to Slynd™ 4 mg tablets. Simulations revealed that a reduction in dose from 4.0 mg to 3.5 mg could be the closer alternative to obtain a point estimate of about 90% for AUC₀₋₇₂ₕ and about 112% for Cₘₐₓ. However, an optimal balance of bioequivalence (BE) compliance for AUC and Cₘₐₓ simultaneously appeared challenging. As shown in the results above, assuming linearity of the system and the PK parameters, an adequate reduction in Cₘₐₓ ratio through reduction of the dose, could result in an inadequate low AUC ratio.

### Conclusion

Based on this data, it was proposed to use DRSP 3.5 mg for further development of a chewable tablet BE to Slynd™ 4 mg tablets.

The following formulations comprising 3.5 mg or 4.0 mg Drospirenone were used for the further trials. Drospirenone features and the method for obtaining thereof are as described in Example 1.

TABLE 4
Quantitative Composition of Drospirenone Chewable Tablets (4.0 mg and 3.5 mg)

| | Chewable Drospirenone | | | | |
|---|---|---|---|---|---|
| | Tablet | | | | |
| | (4 mg) | | Chewable Drospirenone Tablet (3.5 mg) | | |
| | Quantitative | | Quantitative | | Content |
| | Composition | Content | Composition | Content | ranges^{c} |
| Material | (mg) | (% w/w) | (mg) | (% w/w) | (% w/w) |
| Drospirenone ^{a} | 4.00 | 7.05 | 3.5 | 6.17 | 5-10 |
| Microcrystalline | 32.45 | 57.2 | 32.95 | 58.06 | 55-65 |
| cellulose | | | | | |
| Anhydrous | 17.50 | 30.84 | 17.50 | 30.84 | 25-35 |
| Lactose | | | | | |
| Colloidal silicon | 0.29 | 0.51 | 0.29 | 0.51 | 0.1-1 |
| dioxide | | | | | |
| Magnesium | 0.29 | 0.51 | 0.29 | 0.51 | 0.1-2.5 |
| stearate | | | | | |
| Flavoring agent | 0.57 | 1.00 | 0.57 | 1.00 | 0.5-2 |
| Coating agent | 1.65 | 2.91 | 1.65 | 2.91 | 1-5 |
| Purified Water ^{b} | trace | trace | trace | trace | Trace |
| TOTAL | 56.75 | 100.0 | 56.75 | 100.0 | 100.0 |

^{a} Drospirenone non micronized
^{b} Removed during the manufacturing process
^{c}Calculated ranges for suitable chewable drospirenone tablets (3.5 mg)

### Example 3. Dissolution Profiles 4.0 Mg Drospirenone Chewable Tablets With Different Hardness

Considering the previous findings, an investigation on modelling and simulations, including IVIVC, to understand better the best strategy for the development of a chewable tablet was proposed. To understand the impact of chewing in the dissolution profile of drospirenone from the formulation, a biorelevant in vitro dissolution method was developed to mimic the chewing of the tablets.

As stated above, the results from the first clinical study (BE study 1 in Example 1) showed that the bioequivalence criteria for the 4.0 mg chewable tablet with a hardness of 40 N was not met, although similar dissolution profiles were observed in vitro. The inventors hypothesized that the chewing process of the tablets was the main reason to explain the differences between the in vitro dissolution profiles and the clinical results. Thus, additional dissolution methods were investigated in order to simulate the chewing process of the tablets in the mouth in order to try to find a biorelevant dissolution method.

### Use of an Artificial Jaw for Simulating Chewing

As a first approach the tablets were tested in the initially selected conditions recited above but after crushing the tablets by compressing and cutting with artificial jaws and teeth to simulate chewing (one bite chew), applying a force with a maximum value of 250 N. Tablets were placed in the vessel for dissolution testing after being crushed by this method (so called “tested as crushed”).

FIG. 2 shows a comparison of the dissolution profile of the 4.0 mg chewable tablet (whole and crushed) versus Slynd™. Testing conditions: 900 ml of water and 0.6% polysorbate, 75 rpm, apparatus II (paddles) and 37° C.

Based on the differences observed on the dissolution profile of the crushed tablets it became necessary to decrease the dissolution profile of drospirenone in the chewable tablets in order to comply with Cₘₐₓ BE requirement. The influence of tablet's hardness on dissolution profile of the drug was investigated. Hardness was measured using a Dr. Schleuniger Pharmatron MultiTest 50 tablet hardness tester (SOTAX, Aesch, Switzerland). Different compression forces were used to manufacture batches to get 4.0 mg chewable tablets with different hardness. The hardness evaluated were 25 N, 45 N and 65 N.

FIG. 3 discloses dissolution profile of the tablets with the different hardness tested as crushed tablets. Testing conditions: 900 ml of water and 0.6% polysorbate, 75 rpm, apparatus II (paddles) and 37° C. (n=3). Slynd™ 4 mg tablets (tested as whole tablet) was included in the comparison of dissolution behavior.

Based on the results obtained, in combination with the results of the pk study disclosed in example 1, a modelling and simulation approach was performed to define the optimal hardness or combination of hardness/dose to select the best potential candidate (as chewable tablet) to have similar Cₘₐₓ and AUC to the reference (oral tablet to be swallowed, Slynd™).

### Example 4: In Vivo/In Vitro Correlation Models (IVIVC)

In Vivo/In Vitro Correlation Models (IVIVC) were developed as a tool to select the most optimal combination of hardness and dose of the chewable tablet that could met Bioequivalence criteria as compared to the drospirenone 4.0 mg swallowed formulation (Slynd™) before proceeding to the second clinical study. Based on the in vivo data obtained in the clinical study of Example 1 together with previous in-house clinical studies, and the in vitro dissolution data of Example 3 together with previous batches, different IVIVC were developed.

### IVIVC Methodology

The procedure for the development of the IVIVC involved the following steps.
1. Modelling of the mean PK “in vivo” data
2. Modelling of the mean “in vitro” dissolution data
3. Prediction of fraction absorbed in vivo data using a numerical deconvolution approach
4. A single “in vitro-in vivo” correlation (IVIVC) model using the PK data for the different batches used for the IVIVC and their correspondent in vitro dissolution
5. Prediction of the PK profile from the IVIVC and the dissolution profiles at the tested conditions
6. Evaluation of the internal and external predictability of the predicted PK profiles vs the observed mean PK profiles. Good internal and external predictability was obtained using the in vitro results from “crushed” tablets together with data from Slynd™ from previous clinical studies. Therefore, the IVIVC demonstrated that the in vitro model used was simultaneously predictive of the in vivo behavior of the swallowed tablets as well as the chewable tablets.
7. PK prediction of the battery of dissolution profiles to be simulated (combination of different hardness and doses) and comparison of simulated AUC₀₋₇₂ₕ and Cₘₐₓ as compared to Slynd™.

### Results

As result of this evaluation, the prototype with hardness of 65 N at a dose of about 3.6 mg was expected to be the best candidate having PK parameters similar to the reference. It was the one with the closest expected Cₘₐₓ ratio, and limited change on AUC ratio to the reference (Slynd™ batch). As can be observed in FIG. 4A and B, the predictive error was for both AUC₀₋₇₂ₕ and Cₘₐₓ at a dose of 3.6 mg closer to the minimum of 0%. Additionally, a prototype with hardness between 45 N-65 N at a dose of 3.5 mg was expected to be within the acceptance limits for both Cₘₐₓ and AUC.

### Example 5: Assessment of Different Hardness for a 3.5 Mg Drospirenone Chewable Tablet

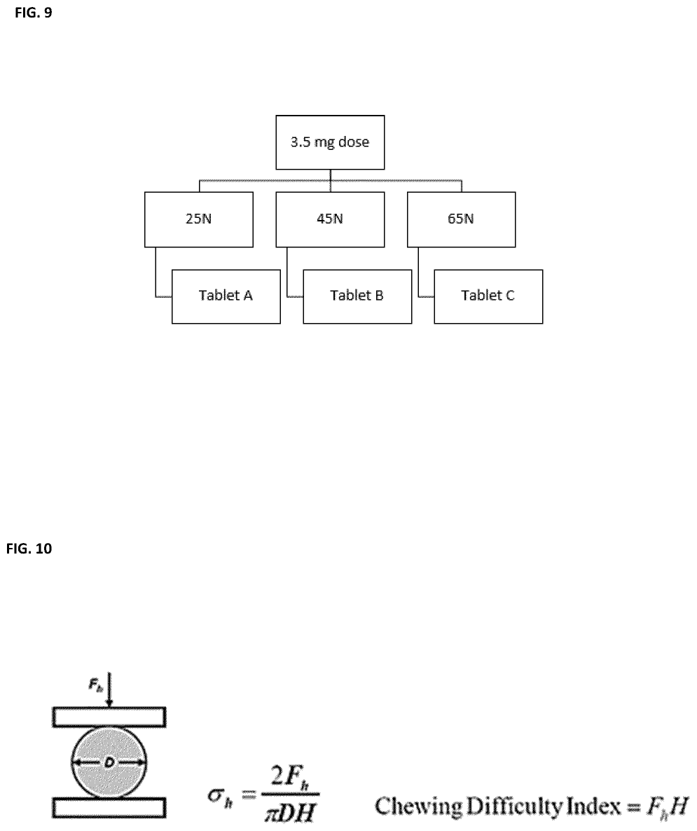

To confirm above conclusions dissolution profile similarity was assessed for the chewable 3.5 mg formulation with different hardness. The influence of different compression forces during the production process in order to get different tablets hardness was evaluated and the dissolution profiles for tablets with different hardness was assessed as depicted in FIG. 9.

The composition of tablets A, B and C products is identical, according to the formulation disclosed in example 2, table 4.

The Dissolution profiles were performed with the crushed tablet versus the reference product Slynd™ using the same conditions as defined in Example 3.

As can be seen in FIG. 5 showing the Dissolution profiles performed crushed versus the reference product Slynd™, the prototype with a hardness of 65 N showed a similar dissolution profile as Slynd™, where the profile follows the same kinetic as the higher dose tablet. As result, a prototype with hardness of 65 N at a dose of about 3.6 mg was expected to have the closest value of Cₘₐₓ with a very limited change on AUC as compared to the swallowed.

### Example 6. Prototype Selection

The final prototype selected for the second clinical study for the chewable formulation was a drospirenone 3.5 mg chewable tablet with a composition as disclosed above in the table 4 and having a core hardness of 50 N (target tablet hardness for the direct compression was set between 40 N and 60 N). After coating, average hardness of the chewable tablets was measured, and found to be about 65 N, more specifically average hardness was 67 N (max 70 N, min 57 N) as shown in Table 5.

TABLE 5
Tablet hardness

Drospirenone 3.5 mg chewable tablets

| Test | Sub batch A | Sub batch B |
|---|---|---|
| Hardness (N) | 64 | 63 |
| | 57 | 68 |
| | 67 | 67 |
| | 64 | 65 |
| | 68 | 63 |
| | 62 | 62 |
| | 62 | 70 |
| | 57 | 70 |
| | 59 | 58 |
| | 65 | 61 |

As expected and due to the aesthetic function of the coating polymer, similar dissolution values were obtained when comparing dissolution profiles from cores and coated tablets (data not shown).

After the prototype was selected, an additional clinical study was performed (Example 7) to evaluate if the selected tandem of hardness and dose for the chewable formulation was bioequivalent to the Slynd™ drug product.

### Example 7: Second Clinical Trial, Bioequivalence Study for Drospirenone 3.5 Mg With a Tablet Hardness of 65 N

Based on previous studies, two different scenarios (prototypes) were presented for performing a bioequivalence study:

Scenario A) Drospirenone 4.0 mg chewable tablet with a hardness of 65 N and a composition as defined in Table 4 above

Scenario B) Drospirenone 3.5 mg chewable tablet with a hardness of 65 N and a composition as defined in Table 4 above

For both scenario 1 and 2, the chewable tablet was placed on the tongue and chewed (break tablet between teeth into small pieces) and swallowed immediately. The subject was asked for confirmation that the tablet had disintegrated and the time when the tablet was completely chewed was recorded. After dosing, the oral cavity was checked.

Population studied: Healthy nonpregnant female volunteers who have previously used hormonal contraceptives without complications, with age≥18 and ≤40 years, body mass index≥18.0 and ≤30.0 kg/m², and non-smokers or ex-smokers were selected according to the defined inclusion and exclusion criteria.

### Clinical Results

### SCENARIO A) Drospirenone Chewable 4.0 mg Results

The clinical results for Drospirenone 4.0 mg chewable tablet obtained were compiled in tables below:

TABLE 6
Chewable 4.0 mg Tablet without water (T) vs Slynd ™Tablets
with water (R) - Fasted

| | T | R | | | |
|---|---|---|---|---|---|
| PK parameter | (n = 39) | (n = 39) | T/R (%) | 90% CI | ISCV % |
| Cₘₐₓ (ng/mL) | 22.35 | 18.03 | 123.94 | 114.49-134.17 | 20.8 |
| AUC₀₋₇₂ | 481.81 | 453.72 | 106.19 | 102.18-110.35 | 10.0 |
| (ng × h/mL) | | | | | |
| Tₘₐₓ (h) | 2.50 | 4.00 | — | — | — |
| | (1.25-6.02) | (2.00-8.00) | | | |

The clinical results for Drospirenone 3.5 mg chewable tablet obtained were compiled in the table below:

TABLE 7
Chewable 3.5 mg Tablet without water (T) vs Slynd ™ Tablets
with water (R)- Fasted

| | T | R | | | |
|---|---|---|---|---|---|
| PK parameter | (n = 37) | (n = 39) | T/R (%) | 90% CI | ISCV % |
| Cₘₐₓ (ng/mL) | 20.58 | 18.08 | 113.81 | 107.07-120.97 | 15.6 |
| AUC₀₋₇₂ | 418.31 | 453.80 | 92.18 | 89.27-95.18 | 8.2 |
| (ng × h/mL) | | | | | |
| Tₘₐₓ (h) | 2.50 | 4.00 | — | — | — |
| | (1.00-4.00) | (2.00-8.00) | | | |

In addition, bioavailability study to assess the effect of food was conducted. The results indicate that the ingestion of a high fat high caloric meal 30 min before the administration of drospirenone 3.5 mg chewable tablet delayed the absorption rate of drospirenone (as assessed by Cₘₐₓ) but showed no significant effect on the extent of absorption (as assessed by AUC₀₋₇₂).

### Conclusions

Drospirenone 3.5 mg tablets with modified hardness meet the BE criteria for Cₘₐₓ and AUC (Acceptance interval of 80.00 to 125.00% for both Cₘₐₓ and AUC₀₋₇₂) with Slynd™ as reference. Modified hardness helps to lower the Cₘₐₓ, but dose adjustment to 3.5 mg is required to meet the BE criteria for Cₘₐₓ. Based on the food effect study, no relevant food effect is observed for either Cₘₐₓ or AUC.

### Example 8: Dosing of Tablet and Tablet Hardness to Comply With BE Requirements

An estimation of the optimal dose (balance between AUC₀₋₇₂ₕ and Cₘₐₓ) expected to be compliant for BE criteria was derived for every hardness from the simulations using IVIVC (see FIG. 6). As can be seen the adjustment of the dosage together with the hardness can lead to the required BE meaning that a suitable chewable tablet can be obtained by combining the hardness of 15 N-100 N with the corresponding amount of drospirenone from 3.4 mg-3.9 mg.

Finally, to in more detail assess the role of the hardness for the dissolution profile, additional prototypes of tablets with a dosage of 3.5 mg drospirenone and with a range of different hardness between 15 N to 100 N were manufactured using the same formulation. The dissolution profiles were prepared by the artificial jaw crushing method described in Example 3 (tested as crushed).

At such conditions, the 3.5 mg drospirenone chewable tablets with a hardness between 30 N-75 N showed a similar dissolution profile to Slynd™ (whole tablet). The dissolution profile mimicking the profile of Slynd™ the closest over the whole time being tablets with a hardness from 50 N-65 N. To be bioequivalent to Slynd™ 4.0 mg tablets, dissolution Tests Specifications for Drospirenone 3.5 mg chewable tablets (tested as crushed) were set as follows:
From 40% to 60% drospirenone is dissolved within 30 minutes
At least 75% drospirenone is dissolved after 240 minutes.

As can be seen in FIG. 7 the 3.5 mg chewable tablets comprising a hardness of between 30 N- 75 N (crushed) comply with the defined specifications.

### Example 9: Chewable 3.5 Mg Tablets Dissolution Profile (Whole Tablet)

Developed method to simulate the chewing process (tested as crushed) was found to provide discriminatory and reproducible in vitro profiles. However, a new dissolution method was developed to tests whole chewable tablets, thus avoid the crushing of the tablet and reducing the variability of the method.

In order to simulate in vitro the chewing process, an additional physical stress was tested by increasing the rotational speed of paddles up to 100 rpm. The increase in the rotational speed up to 100 rpm is the adaptation of the chewing process, and fulfil the following objectives:
To be biorelevant, and to be a reproducible method
To maintain enough discriminatory power
To decrease variability to ensure batch-to-batch consistency

Final testing conditions for the in vitro dissolution testing for chewable tablets (tested as whole tablets) is set as follows:
Dissolution Medium: water with 0.6% polysorbate
Volume: 900 mL
Rotational speed: 100 rpm
Type: apparatus 2 (paddles)
Temperature: 37° C.±0.5° C.

FIG. 8 shows the comparative dissolution profiles in QC media (900 mL water, 0.6% polysorbate, apparatus 2 (paddles), 100 rpm) for Slynd™ 4 mg tablets and drospirenone 3.5 mg chewable tablets.

Using these conditions, drospirenone 3.5 mg chewable tablets were found to have a slower dissolution profile than Slynd™ (Drospirenone 4 mg tablets). The inventors thus defined 30 min as the first timepoint and 240 min as the last timepoint. To be bioequivalent to Slynd™

(Drospirenone 4 mg tablets), specifications for drospirenone 3.5 mg chewable tablets in the newly developed in vitro dissolution test were set as follows:
From 20% to 40% drospirenone is dissolved within 30 minutes
At least 75% drospirenone is dissolved after 240 minutes.

### Example 10: Assessment of Optimal Size for Chewable Tablet

Following the Guidance for industry, the chewable tablets of the invention were characterized for its chewable properties including hardness, disintegration, and dissolution, as well as all factors that may influence drug bioavailability and bioequivalence. The above-mentioned Critical Quality Attributes (CQA) considerations have been conducted and evaluated on to-be- marketed product Drospirenone 3.5 mg Chewable Tablets. Results are described in table 8.

TABLE 8
Physical characterization of Drospirenone
3.5 mg Chewable Tablets

| | Product | | |
|---|---|---|---|
| | Drospirenone 3.5 mg Chewable Tablets | | |
| | Batches | | |
| | #01 | #02 | #03 |
| Hardness (N) | 66N | 67N | 65N |
| Disintegration time | 6 | 5 | 3 |
| in water (min) | | | |
| Chewing difficulty | 158.4N mm | 160.8N mm | 162.5N mm |
| index (CDI) (N*mm) | | | |

The chewable tablets have been evaluated for the chewing difficulty index. Two methods were used for measuring tablet strength: diametral compression (diametrical tensile strength) and flexural bending (flexure tensile strength test). The relationship between the two methods is explored to calculate a value which is described here as a chewing difficulty index according to the formula depicted in FIG. 10.

“Fh” is the load required to break the tablet, “D” is the tablet diameter and “H” is the tablet thickness. The variation of those parameters is related to hardness and size of the tablets. As the differences in hardness, diameter and thickness of the tablets is minimum, both parameters are practically the same for the three batches. Based on these results, it can be demonstrated that the Drospirenone 3.5 mg chewable tablets, could be easily chewable and swallowed. The tablets with different diameters and thicknesses showed the following surface areas (SSA) and volumes (V). The shaded boxes show the values for surface area and volume that are not beyond the acceptable ranges according to industry guidelines:

| | DIAMETER | | | | | |
|---|---|---|---|---|---|---|
| | (5-6 mm) | | | | | |
| THICKNESS | 5 mm | | 5.5 mm | | | |
| (1.9-2.9 mm) | SSA | V | SSA | V | 6 mm | |
| mm | (mm2) | (mm3) | (mm2) | (mm3) | SSA | V |
| 1.9 | 56.87 | 29.66 | 62.94 | 31.25 | 79.60 | 43.26 |
| 2.0 | 58.44 | 31.62 | 64.67 | 33.62 | 81.49 | 46.09 |
| 2.1 | 60.01 | 33.59 | 66.39 | 36.00 | 83.37 | 48.91 |
| 2.2 | 61.58 | 35.55 | 68.12 | 38.37 | 85.26 | 51.74 |
| 2.3 | 63.15 | 37.52 | 69.85 | 40.75 | 87.14 | 54.57 |
| 2.4 | 64.73 | 39.48 | 71.58 | 43.12 | 89.03 | 57.39 |
| 2.5 | 66.30 | 41.44 | 73.31 | 45.50 | 90.91 | 60.22 |
| 2.6 | 67.87 | 43.41 | 75.03 | 47.87 | 92.80 | 63.05 |
| 2.7 | 69.44 | 45.37 | 76.76 | 50.25 | 94.68 | 65.87 |
| 2.8 | 71.01 | 47.34 | 78.49 | 52.62 | 96.57 | 68.70 |
| 2.9 | 72.58 | 49.30 | 80.22 | 55.00 | 98.45 | 71.53 |

### Example 11: Manufacturing and Testing of in Vitro Dissolution Profile of Further Formulations

Tablets according to the invention comprising 3.5 mg drospirenone were manufactured by direct blend followed by tableting with the compression force indicated in each case. Testing conditions for the in vitro dissolution testing for the chewable tablets obtained (tested as whole tablets):
Dissolution Medium: water with 0.6% polysorbate
Volume: 900 mL
Rotational speed: 100 rpm
Type: apparatus 2 (paddles)
Temperature: 37° C.±0.5° C.

### 1. Formulations With Different Filler, 50 N

| | Trial 1 | | Trial 2 | | Trial 3 | |
|---|---|---|---|---|---|---|
| | Content | Content | Content | Content | Content | Content |
| Component | (mg) | (% w/w) | (mg) | (% w/w) | (mg) | (% w/w) |
| Drospirenone | 3.50 | 6.35 | 3.5 | 6.35 | 3.5 | 6.35 |
| (non micronized, | | | | | | |
| D₅₀ = 31.03 μm) | | | | | | |
| Microcrystalline cellulose | 32.95 | 59.80 | 32.95 | 59.80 | 32.95 | 59.80 |
| Starch DC | 17.50 | 31.76 | — | — | — | — |
| Mannitol | — | — | 17.50 | 31.76 | — | — |
| Calcium carbonate | — | — | — | — | 17.50 | 31.76 |
| Colloidal silicon Dioxide | 0.29 | 0.53 | 0.29 | 0.53 | 0.29 | 0.53 |
| Magnesium stearate | 0.29 | 0.53 | 0.29 | 0.53 | 0.29 | 0.53 |
| Flavoring agent | 0.57 | 1.03 | 0.57 | 1.03 | 0.57 | 1.03 |
| TOTAL (mg) | 55.10 | 100 | 55.10 | 100 | 55.10 | 100 |

| | % of DRSP dissolved | % of DRSP dissolved |
|---|---|---|
| Trial | within 30 min | within 4 h |
| 1 | 30 | 81 |
| 2 | 26 | 75 |
| 3 | 23 | 75 |

### 2. Formulations With Different Binder, 50 N

| | Trial 5 | | Trial 6 | |
|---|---|---|---|---|
| | Content | Content | Content | Content |
| Component | (mg) | (% w/w) | (mg) | (% w/w) |
| Drospirenone (non | 3.50 | 6.35 | 3.5 | 6.35 |
| micronized, D₅₀ = 31.03 μm) | | | | |
| Lactose | 17.50 | 31.76 | 17.50 | 31.76 |
| Methycellulose | 32.95 | 59.80 | — | — |
| Povidone | — | — | 32.95 | 59.80 |
| Colloidal silicon Dioxide | 0.29 | 0.53 | 0.29 | 0.53 |
| Magnesium stearate | 0.29 | 0.53 | 0.29 | 0.53 |
| Flavoring agent | 0.57 | 1.03 | 0.57 | 1.03 |
| TOTAL (mg) | 55.10 | 100 | 55.10 | 100 |

| | % of DRSP dissolved | % of DRSP dissolved |
|---|---|---|
| Trial | within 30 min | within 4 h |
| 5 | 33 | 84 |
| 6 | 33 | 89 |

### 3. Formulations With Different Lubricant.50 N

| | Trial 8 | | Trial 9 | |
|---|---|---|---|---|
| | Content | Content | Content | Content |
| Component | (mg) | (% w/w) | (mg) | (% w/w) |
| Drospirenone (non | 3.50 | 6.35 | 3.5 | 6.35 |
| micronized, D₅₀ = 31.03 μm) | | | | |
| Microcrystalline cellulose | 32.95 | 59.80 | 32.95 | 59.80 |
| Lactose | 16.66 | 30.24 | 16.37 | 29.71 |
| Colloidal silicon Dioxide | 0.29 | 0.53 | 0.29 | 0.53 |
| Sodium dodecyl sulfate | 1.13 | 2.05 | — | — |
| Polyethylene glycol 6000 | — | — | 1.42 | 2.58 |
| Flavoring agent | 0.57 | 1.03 | 0.57 | 1.03 |
| TOTAL (mg) | 55.10 | 100 | 55.1 | 100 |

| | % of DRSP dissolved | % of DRSP dissolved |
|---|---|---|
| Trial | within 30 min | within 4 h |
| 8 | 32 | 79 |
| 9 | 27 | 77 |

### 4. Formulation 100 N. Micronized Drospirenone

Trial 12

| | Content | Content |
|---|---|---|
| Component | (mg) | (% w/w) |
| Drospirenone (micronized D₉₀ = 5.64 μm) | 3.50 | 6.35 |
| Microcrystalline cellulose | 32.95 | 59.80 |
| Lactose | 17.50 | 31.76 |
| Colloidal silicon Dioxide | 0.29 | 0.53 |
| Magnesium stearate | 0.29 | 0.53 |
| Flavoring agent | 0.57 | 1.03 |
| TOTAL (mg) | 55.10 | 100 |

| | % of DRSP dissolved | % of DRSP dissolved |
|---|---|---|
| Trial | within 30 min | within 4 h |
| 12 | 21 | 87 |

### 5. Formulation 100 N With Disintegrant

| | Trial 13 | |
|---|---|---|
| | Content | Content |
| Component | (mg) | (% w/w) |
| Drospirenone (non micronized, D₅₀ = 31.03 μm) | 3.50 | 6.35 |
| Microcrystalline cellulose | 32.38 | 58.77 |
| Lactose | 17.50 | 31.76 |
| Crospovidone | 0.57 | 1.03 |
| Colloidal silicon Dioxide | 0.29 | 0.53 |
| Magnesium stearate | 0.29 | 0.53 |
| Flavoring agent | 0.57 | 1.03 |
| TOTAL (mg) | 55.10 | 100 |

| | % of DRSP dissolved | % of DRSP dissolved |
|---|---|---|
| Trial | within 30 min | within 4 h |
| 13 | 22 | 75 |

### 6. Formulation 15 N+Matrix Retarding Agent

| | Trial 15 | |
|---|---|---|
| | Content | Content |
| Component | (mg) | (% w/w) |
| Drospirenone (non micronized, D₅₀ = 31.03 μm) | 3.50 | 6.21 |
| Microcrystalline cellulose | 28.52 | 50.62 |
| Lactose | 17.50 | 31.06 |
| HPMC (Methocel K100) | 5.67 | 10.06 |
| Colloidal silicon Dioxide | 0.29 | 0.52 |
| Magnesium stearate | 0.29 | 0.52 |
| Flavoring agent | 0.57 | 1.01 |
| TOTAL (mg) | 56.34 | 100 |

| | % of DRSP dissolved | % of DRSP dissolved |
|---|---|---|
| Trial | within 30 min | within 4 h |
| 15 | 39 | 82 |

### 7. Different Combinations Excipients 50 N

| | Trial 18 | |
|---|---|---|
| | Content | Content |
| Component | (mg) | (% w/w) |
| Drospirenone (non micronized, D₅₀ = 31.03 μm) | 3.50 | 6.29 |
| Lactose | 32.38 | 58.17 |
| Mannitol | 16.66 | 29.94 |
| Talc | 0.57 | 1.02 |
| Croscarmellose | 1.13 | 2.03 |
| Magnesium stearate | 0.85 | 1.53 |
| Flavoring agent | 0.57 | 1.02 |
| TOTAL (mg) | 55.66 | 100 |

| | % of DRSP dissolved | % of DRSP dissolved |
|---|---|---|
| Trial | within 30 min | within 4 h |
| 18 | 35 | 80 |

| | Trial 19 | |
|---|---|---|
| | Content | Content |
| Component | (mg) | (% w/w) |
| Drospirenone (non micronized, D₅₀ = 31.03 μm) | 3.50 | 6.35 |
| Lactose | 17.50 | 31.77 |
| Gelatin | 31.81 | 57.74 |
| Colloidal silicon Dioxide | 0.29 | 0.53 |
| Croscarmellose | 1.13 | 2.05 |
| Magnesium stearate | 0.29 | 0.53 |
| Flavoring agent | 0.57 | 1.03 |
| TOTAL (mg) | 55.10 | 100 |

| | % of DRSP dissolved | % of DRSP dissolved |
|---|---|---|
| Trial | within 30 min | within 4 h |
| 19 | 32 | 84 |

| | Trial 20 | |
|---|---|---|
| | Content | Content |
| Component | (mg) | (% w/w) |
| Drospirenone (non micronized, D₅₀ = 31.03 μm) | 3.50 | 6.35 |
| Starch DC | 17.50 | 31.77 |
| Povidone | 31.81 | 57.74 |
| Colloidal silicon Dioxide | 0.29 | 0.53 |
| Croscarmellose | 1.13 | 2.05 |
| Magnesium stearate | 0.29 | 0.53 |
| Flavoring agent | 0.57 | 1.03 |
| TOTAL (mg) | 55.1 | 100 |

| | % of DRSP dissolved | % of DRSP dissolved |
|---|---|---|
| Trial | within 30 min | within 4 h |
| 20 | 35 | 90 |

| | Trial 21 | |
|---|---|---|
| | Content | Content |
| Component | (mg) | (% w/w) |
| Drospirenone (non micronized, D₅₀ = 31.03 μm) | 3.50 | 6.33 |
| Lactose | 17.68 | 32.00 |
| Microcrystalline cellulose | 31.14 | 56.34 |
| Talc | 2.27 | 4.10 |
| Sodium dodecyl sulfate | 0.11 | 0.20 |
| Flavoring agent | 0.57 | 1.03 |
| TOTAL (mg) | 55.27 | 100 |

| | % of DRSP dissolved | % of DRSP dissolved |
|---|---|---|
| Trial | within 30 min | within 4 h |
| 21 | 28 | 79 |

### Items of the Invention

The present invention relates to the following items:

An oral chewable tablet comprising 3.4 mg-3.7 mg, preferably 3.5 mg of drospirenone, wherein the tablet has a dissolution profile characterized in that no more than 20-40%, of the drospirenone initially present in said composition is dissolved within 30 minutes when the composition is subjected to an in vitro dissolution test in 900 ml of water with 0.6% polysorbate using USP apparatus 2 (paddles) at 100 rpm, preferably at a temperature of 37° C.±0.5° C.; the percentages of drospirenone being related to the amount of drospirenone initially present in said composition.

In a preferred embodiment at least 75% of said drospirenone is dissolved after 4 hours.

In a preferred embodiment at least 50%, preferably at least 60%, 65% or 70% of said drospirenone is dissolved in a time range from about 3 hours to about 4 hours.

In another preferred embodiment at least 70%, at least 71%, at least 72%, at least 73%, at least 74%, at least 75%, at least 76%, at least 77%, at least 78%, at least 79%, at least 80% of said drospirenone is dissolved after 4 hours.

The subject-matter in the above embodiments can be combined with any of the embodiments described herein below.

An oral chewable tablet comprising 3.4 mg-3.7 mg, preferably 3.5 mg of drospirenone, wherein said tablet further has a hardness of 15 N-100 N, preferably from 30 N-75 N, more preferably of 40 N-65 N, wherein the tablet does not comprise any estrogen.

An oral chewable tablet comprising from 3.4 mg-3.7 mg, preferably 3.5 mg drospirenone, wherein the tablet is formulated such that the tablet provides a mean Cₘₐₓ of less than 30 ng/ml drospirenone, preferably less than 25 ng/ml in a subject when the tablet is chewed or disintegrated in the oral cavity of the subject prior to swallowing.

The oral chewable tablet as described in the items above, wherein the tablet does not comprise any estrogen.

The oral chewable tablet as described in the items above, said tablet comprising drospirenone as the only contraceptive ingredient.

The oral chewable tablet as described in the items above, wherein said drospirenone has a d50 particle size ranging from 10 μm to 60 μm, preferably a d50 particle size ranging from 20 μm to 50 μm.

The oral chewable tablet as described in the items above, the tablet further comprising one or more pharmaceutically acceptable excipients.

The oral chewable tablet as described in the items above, wherein the said pharmaceutically acceptable excipients are at least one binder and at least one filler, and wherein:
(i) the amount of drospirenone accounts for 1% to 10% by weight
(ii) the amount of the at least one binder accounts for 50% to 65% by weight and
(iii) the amount of the at least one filler accounts for 25% to 35% by weight, the percentages by weight being related to the total weight of the said tablet.

The oral chewable tablet as described in the items above, further comprising at least one glidant and at least one lubricant wherein:
(iv) the amount of the at least one glidant accounts for 0.2% to 6% by weight and
(v) the amount of the at least one lubricant accounts for 0.2% to 2.5% by weight the percentages by weight being related to the total weight of the said tablet.

The oral chewable tablet as described in the items above, wherein:
(i) the at least one binder is microcrystalline cellulose
(ii) the at least one filler is anhydrous lactose
(iii) the at least one glidant is silicon dioxide and
(iv) the at least one lubricant is magnesium stearate.

The oral chewable tablet as described in the items above, wherein said tablet comprises or consists of:
a) 3.4 mg-3.7 mg of drospirenone, preferably 3.5 mg of drospirenone;
b) at least one binder at 55% to 65% w/w, preferably about 60% w/w;
c) at least one filler at 25% to 35% w/w, preferably about 30% w/w;
d) at least one glidant 0.1% to 1% w/w; preferably about 0.5% w/w;
e) at least one lubricant 0.1% to 2.5% w/w; preferably about 0.5% w/w; and
f) optionally a flavouring agent 0.5% to 2% w/w; preferably about 1% w/w. the percentages by weight being related to the total weight of the said tablet.

The oral chewable tablet as described in the items above, wherein said tablet comprises:
a) 3.4 mg-3.7 mg of drospirenone, preferably 3.5 mg of drospirenone;
b) microcrystalline cellulose 55% to 65% w/w; preferably about 60% w/w;
c) anhydrous lactose 25% to 35% w/w; preferably about 30% w/w;
d) silicon dioxide 0.1% to 1% w/w; preferably about 0.5% w/w;
e) magnesium stearate 0.1% to 2.5% w/w; preferably about 0.5% w/w; and
f) optionally, a flavouring agent 0.5% to 2% w/w; preferably about 1% w/w.

The oral chewable tablet as described in the items above, wherein said tablet further comprises at least one sweetener and/or at least one colour agent.

The oral chewable tablet as described in the items above, wherein said tablet has a round shape and a diameter of between 4.0 to 6.0 mm, more preferred of 4.5 to 5.5 mm, most preferred of 5.0 mm.

Use of the oral chewable tablet as described in the items above for providing contraception to an individual.

Use of the oral chewable tablet as described in the item above, comprising administering one tablet once daily to said individual.

A method for providing contraception to an individual, comprising orally administering the oral chewable tablet as described in the items above to the individual, wherein the individual chews or disintegrates the tablet in the oral cavity of the subject prior to swallowing.

A kit, preferably a contraceptive kit, comprising one or more packaging units, wherein each packaging unit comprises 24 to 28 active daily dosage units, each daily dosage unit comprising an oral chewable tablet as described in the items above.

A method for providing contraception in a subject, comprising administering to the subject an oral dosage form comprising from 3.4 mg-3.7 mg, preferably 3.5 mg drospirenone, wherein the oral dosage form is chewed or disintegrated in the oral cavity of the subject prior to swallowing, and wherein the administering results in a mean Cₘₐₓ of less than 25 ng/ml drospirenone in the subject after a single administration of the oral dosage form.

The method as described in the item above, wherein the oral dosage form does not comprise any estrogen, preferably, wherein drospirenone is the only active ingredient of the oral dosage form.

The method of as described in the items above, wherein the oral dosage form is a chewable oral tablet as described in the items above.

1. An oral chewable tablet comprising 3.4 mg-3.7 mg, preferably 3.5 mg of drospirenone, wherein the tablet has a dissolution profile characterized in that no more than 20-40%, of the drospirenone initially present in said composition is dissolved within 30 minutes when the composition is subjected to an in vitro dissolution test in 900 ml of water with 0.6% polysorbate using USP apparatus 2 (paddles) at 100 rpm, preferably at a temperature of 37° C.±0.5° C.; the percentages of drospirenone being related to the amount of drospirenone initially present in said composition.

2. An oral chewable tablet comprising 3.4 mg-3.7 mg, preferably 3.5 mg of drospirenone, wherein said tablet further has a hardness of 15 N-100 N, preferably 30 N-75 N, more preferably of 40 N-65 N, wherein the tablet does not comprise any estrogen.

3. An oral chewable tablet comprising from 3.4 mg-3.7 mg, preferably 3.5 mg of drospirenone, wherein the tablet is formulated such that the tablet provides a mean Cₘₐₓ of less than 30 ng/ml drospirenone, preferably of less than 25 ng/ml in a subject after a single administration, when the tablet is chewed or disintegrated in the oral cavity of the subject prior to swallowing.

4. The oral chewable tablet of any of claims 1 to 3, wherein the tablet does not comprise any estrogen, preferably wherein said tablet comprises drospirenone as the only contraceptive ingredient.

5. The oral chewable tablet according to any one of the preceding claims, wherein said drospirenone has a d50 particle size ranging from 10 μm to 60 μm, preferably a d50 particle size ranging from 20 μm to 50 μm.

6. The oral chewable tablet according to any one of the preceding claims, the tablet further comprising one or more pharmaceutically acceptable excipients.

7. The oral chewable tablet according to claim 6, wherein the said pharmaceutically acceptable excipients are at least one binder and at least one filler, and wherein:
(i) the amount of drospirenone accounts for 1% to 10% by weight,
(ii) the amount of the at least one binder accounts for 50% to 65% by weight and
(iii) the amount of the at least one filler accounts for 25% to 35% by weight,
the percentages by weight being related to the total weight of the said tablet.

8. The oral chewable tablet of claim 7, further comprising at least one glidant and at least one lubricant wherein:
(i) the amount of the at least one glidant accounts for 0.2% to 6% by weight and
(ii) the amount of the at least one lubricant accounts for 0.2% to 2.5% by weight,
the percentages by weight being related to the total weight of the said tablet.

9. The oral chewable tablet according to any of the preceding claims, wherein said tablet comprises or consists of:
a) 3.4 mg-3.7 mg of drospirenone, preferably 3.5 mg of drospirenone;
b) at least one binder at 55% to 65% w/w, preferably about 60% w/w;
c) at least one filler at 25% to 35% w/w, preferably about 30% w/w;
d) at least one glidant 0.1%-1% w/w; preferably about 0.5% w/w;
e) at least one lubricant 0.1%-2.5% w/w; preferably about 0.5% w/w; and
f) optionally a flavouring agent 0.5%-2% w/w; preferably about 1% w/w.
the percentages by weight being related to the total weight of the said tablet.

10. The oral chewable tablet according to any of the preceding claims, wherein:
(i) the at least one binder is microcrystalline cellulose,
(ii) the at least one filler is anhydrous lactose,
(iii) the at least one glidant is silicon dioxide and
(iv) the at least one lubricant is magnesium stearate.

11. The oral chewable tablet according to any of the preceding claims, wherein said tablet comprises or consists of:
a) 3.4 mg-3.7 mg of drospirenone, preferably 3.5 mg of drospirenone;
b) microcrystalline cellulose 55% to 65% w/w; preferably about 60% w/w;
c) anhydrous lactose 25% to 35% w/w; preferably about 30% w/w;
d) silicon dioxide 0.1% to 1% w/w; preferably about 0.5% w/w;
e) magnesium stearate 0.1% to 2.5% w/w; preferably about 0.5% w/w; and
f) optionally, a flavouring agent 0.5% to 2% w/w; preferably about 1% w/w,
the percentages by weight being related to the total weight of the said tablet.

12. The oral chewable tablet according to any one of the preceding claims, wherein said tablet has a maximum diameter of between 4.0 to 6.0 mm, more preferred of 4.5 to 5.5 mm, most preferred of 5.0 mm.

13. The oral chewable tablet according to any one of the preceding claims, wherein said tablet has a thickness of between 1.9 mm to 3.6 mm, more preferred of between 2.1 mm to 3.6 mm, most preferred of 2.4 mm.

14. Use of the oral chewable tablet according to any one of the preceding claims for providing contraception to an individual.

15. Use of the oral chewable tablet of claim 14, comprising administering one tablet once daily to said individual.

16. A kit, preferably a contraceptive kit, comprising one or more packaging units, wherein each packaging unit comprises 24 to 28 active daily dosage units, each daily dosage unit comprising an oral chewable tablet according to any one of the preceding claims.
